(19) United States
(12) Patent Application Publication
(10) Pub. No.: US 2025/0137038 A1
(43) Pub. Date: May 1, 2025

(54) SENSITIVITY AND ESTIMATION OF TUMOR-INFORMED MINIMAL RESIDUAL DISEASE PANELS

(71) Applicant: Myriad Women's Health, Inc., South San Francisco, CA (US)

(72) Inventors: Ashley Acevedo, South San Francisco, CA (US); Genevieve Gould, South San Francisco, CA (US); Christopher J. Battey, South San Francisco, CA (US)

(73) Assignee: Myriad Women's Health, Inc., South San Francisco, CA (US)

(21) Appl. No.: 18/930,353

(22) Filed: Oct. 29, 2024

Related U.S. Application Data
(60) Provisional application No. 63/546,467, filed on Oct. 30, 2023.

(51) Int. Cl. C12Q 1/6827; C12N 15/10; G16B 35/20

(52) U.S. Cl. CPC C12Q 1/6827; C12N 15/1093; G16B 35/20

(57) ABSTRACT

Described herein are methods for improving sensitivity and estimation of tumor-informed MRD assays and reducing error rates by performing post-sequencing error-correction and error correction using internal controls that are proximate to the variant sites.

### CROSS-REFERENCE TO RELATED APPLICATIONS

This application claims the benefit under 35 U.S.C. § 119 (e) of U.S. Provisional Patent Application No. 63/546,467, filed Oct. 30, 2023, the entire contents of which is incorporated herein by reference in its entirety.

### TECHNICAL FIELD

Described herein, are methods of improving the sensitivity and estimation of tumor-informed minimal residual disease (MRD) assays and panels which reduce context-specific background error rates.

### BACKGROUND

The following description of the background of the present technology is provided simply as an aid in understanding the present technology and is not admitted to describe or constitute prior art to the present technology.

The discovery of cell free deoxyribonucleic acid has promoted the non-invasive detection of alterations in genomic sequences that occur in various disease states. However, in some instances, e.g., cancer, the ability to determine the presence of disease by detecting disease-associated mutations has been hindered by the extremely low levels of cell free tumor DNA. Methods that allow for the accurate detection of disease-associated mutations remain desirable. In addition, there also remains a need for the determination of tumor fraction in pre- and post-treatment cancer patients.

### SUMMARY

The present disclosure provides methods for improving sensitivity and estimation of tumor-informed MRD assays and reducing error rates by performing post-sequencing error-correction and error correction using internal controls that are proximate to the variant sites or anywhere within the sequence library or in any sequence reads.

In one aspect, the present disclosure provides methods of detecting circulating tumor DNA (ctDNA) in a sample, comprising: (a) obtaining a tumor sample and a non-tumor sample from a cancer patient; (b) sequencing DNA from the tumor sample and sequencing DNA from the non-tumor sample, thereby obtaining sequences of DNA from the tumor sample and sequences of DNA from the non-tumor sample; (c) determining somatic variants based on differences between sequences of DNA from the tumor sample and sequences of DNA from the non-tumor sample; (d) at a later time, obtaining at least one further sample of blood, plasma, or serum from the cancer patient; (e) extracting cell-free DNA (cfDNA) from the at least one further sample; (f) sequencing the cfDNA, thereby obtaining a sequencing library; and (g) detecting the presence or absence of ctDNA sequences in the sequencing library, wherein detecting the presence or absence of ctDNA sequences in the sequencing library comprises: (i) calculating, using a computer processor, an allele balance of the somatic variants based on purity of the tumor sample obtained from the subject, (ii) calculating, using a computer processor, a copy number for each somatic variants, (iii) calculating, using a computer processor, an error rate for each somatic variant, or (iv) any combination thereof.

For the purposes of any of the foregoing aspects or embodiments, methods of detecting ctDNA in a sample further comprises calculating, using a computer processor, a dropout rate of sites that do not contribute to a ctDNA signal.

For the purposes of any of the foregoing aspects or embodiments, methods of detecting ctDNA in a sample further comprises correcting for, using a computer processor, inclusion of latent variant classes in the sequencing library by comparing the sequence library to a reference panel (e.g., known buffy coat sequences).

For example, detecting the presence or absence of ctDNA sequences in the sequencing library may comprise calculating, using a computer processor, an allele balance of the somatic variants based on purity of the tumor sample obtained from the subject. Alternatively, detecting the presence or absence of ctDNA sequences in the sequencing library may comprise calculating, using a computer processor, a copy number for each somatic variant. Alternatively, detecting the presence or absence of ctDNA sequences in the sequencing library may comprise calculating, using a computer processor, an error rate for each somatic variant. Alternatively, detecting the presence or absence of ctDNA sequences in the sequencing library may comprise calculating, using a computer processor, a dropout rate of sites that do not contribute to a ctDNA signal. Alternatively, detecting the presence or absence of ctDNA sequences in the sequencing library may comprise correcting for, using a computer processor, inclusion of latent variant classes in the sequencing library by comparing the sequence library to a reference panel. Alternatively, detecting the presence or absence of ctDNA sequences in the sequencing library may comprise calculating, using a computer processor, an allele balance of the somatic variants based on purity of the tumor sample obtained from the subject; calculating, using a computer processor, a copy number for each somatic variant; and calculating, using a computer processor, an error rate for each somatic variant. And finally, detecting the presence or absence of ctDNA sequences in the sequencing library may comprise calculating, using a computer processor, an allele balance of the somatic variants based on purity of the tumor sample obtained from the subject; calculating, using a computer processor, a copy number for each somatic variant; calculating, using a computer processor, an error rate for each somatic variant; calculating, using a computer processor, a dropout rate of sites that do not contribute to a ctDNA signal; and correcting for, using a computer processor, inclusion of latent variant classes in the sequencing library by comparing the sequence library to a reference panel.

In another aspect, the present disclosure provides methods of detecting circulating tumor DNA (ctDNA) in a sample comprising: (a) obtaining a tumor sample and a non-tumor sample from a cancer patient; (b) sequencing DNA from the tumor sample and sequencing DNA from the non-tumor sample, thereby obtaining sequences of DNA from the tumor sample and sequences of DNA from the non-tumor sample; (c) determining somatic variants based on differences between sequences of DNA from the tumor sample and sequences of DNA from the non-tumor sample; (d) at a later time, obtaining at least one further sample of blood, plasma, or serum from the cancer patient; (e) extracting cell-free DNA (cfDNA) from the at least one further sample; (f) sequencing the cfDNA, thereby obtaining a sequencing library; (g) selecting a control site in the sequence library for each somatic variant, wherein the control site matches a reference base for the corresponding somatic variant; (h) calculating, using a computer processor, an error rate for a variant allele of the reference base by detecting changes at the control site, wherein the error rate is a background probability of detecting the variant allele; and (i) detecting the presence or absence of ctDNA sequences in the sequencing library. For the purposes of this aspect, the control site(s) can be anywhere within the sequence library or in any sequence reads. However, in some embodiments, the control site is located within about 160 bases of the corresponding somatic variant. In some embodiments, the control site is located within about 120 bases of the corresponding somatic variant. In some embodiments, the control site is located within about 20 bases of the somatic variant. In some embodiments, the control site is located within about 3 bases of the corresponding somatic variant.

In another aspect, the present disclosure provides methods of detecting circulating tumor DNA (ctDNA) in a sample, comprising: (a) obtaining a tumor sample and a non-tumor sample from a cancer patient; (b) sequencing DNA from the tumor sample and sequencing DNA from the non-tumor sample, thereby obtaining sequences of DNA from the tumor sample and sequences of DNA from the non-tumor sample; (c) determining somatic variants based on differences between sequences of DNA from the tumor sample and sequences of DNA from the non-tumor sample; (d) at a later time, obtaining at least one further sample of blood, plasma, or serum from the cancer patient; (e) extracting cell-free DNA (cfDNA) from the at least one further sample; (f) sequencing the cfDNA, thereby obtaining a sequencing library; and (g) detecting the presence or absence of ctDNA sequences in the sequencing library, wherein ctDNA is detected when an enriched somatic variant is detected at a higher rate than at a corresponding control site, and ctDNA is absent when (h) the enriched somatic variant is detected at a higher rate at the corresponding control site compared to the somatic variant site, (ii) the enriched somatic variant is not detected and the corresponded control site is detected, or (iii) neither the enriched somatic variant nor the corresponding control site are detected. For the purposes of this aspect, the corresponding control site for each somatic variant is located within 20, 19, 18, 17, 16, 15, 14, 13, 12, 11, 10, 9, 8, 7, 6, 5, 4, 3, 2, or 1 bases if the somatic variant. In some embodiments, the corresponding control site for each somatic variant is located within about 3 bases of the somatic variant on the DNA fragment. In some embodiments, the control site(s) can be anywhere within the sequence library or in any sequence reads.

For the purposes of any of the foregoing aspects or embodiments, the methods may further comprise enriching the extracted cfDNA by contacting the extracted cfDNA with a plurality of oligonucleotides, wherein each oligonucleotide in the plurality of oligonucleotides comprises a nucleic acid sequence that is capable of hybridizing to a DNA fragment comprising one of the somatic variants, thereby obtaining a ctDNA-enriched fraction. In some embodiments, enriching the extracted cfDNA comprises contacting the extracted cfDNA with a plurality of oligonucleotides, wherein each oligonucleotide in the first plurality of oligonucleotides comprises a nucleic acid sequence that is capable of hybridizing to a DNA fragment comprising one of the somatic variants and a corresponding control site, wherein the corresponding control site for each somatic variant is located within 20 bases of the somatic variant on the DNA fragment, and wherein the corresponding control site optionally comprises a reference base that is that same as the base of the somatic variant. For the purposes of any embodiment involving enriching cfDNA prior to sequencing, enriching may comprise (i) hybrid capture-based enrichment, (ii) PCR-target enrichment, or (iii) on-sequencer enrichment.

For the purposes of any of the foregoing aspects or embodiments, sequencing may comprise whole genome sequencing or targeted sequencing, such as sequencing of introns, exons, intergenic regions, or a combination thereof.

For the purposes of any of the foregoing aspects or embodiments, the somatic variants may comprise at least 10, at least 50, at least 100, at least 150, at least 200, at least 250, at least 500, at least 750, at least 1000, at least 1100, at least 1200, at least 1300, at least 1400, at least 1500, at least 1600, at least 1700, at least 1800, at least 1900, or at least 2000 tumor-specific somatic mutations.

For the purposes of any of the foregoing aspects or embodiments, the somatic variants may comprise one or more somatic mutations selected from SNVs, insertions, deletions, and translocations.

For the purposes of any of the foregoing aspects or embodiments, the methods may further comprise determining a tumor fraction. In some embodiments, a tumor fraction of zero indicates the absence of the tumor in the patient.

For the purposes of any of the foregoing aspects or embodiments, the tumor sample comprises a solid tumor biopsy or a fluid sample, such as blood, blood plasma, blood serum, urine, saliva, and cerebral spinal fluid (CSF).

For the purposes of any of the foregoing aspects or embodiments, the non-tumor sample comprises a tissue sample matched to a tissue of origin of the tumor sample. Alternatively, for the purposes of any of the foregoing aspects or embodiments, the non-tumor sample comprises a fluid sample selected from a buffy coat sample, blood, blood plasma, blood serum, urine, saliva, and cerebral spinal fluid (CSF).

For the purposes of any of the foregoing aspects or embodiments, the patient may have completed at least one cancer treatment prior to obtaining the tumor sample and the non-tumor sample. The cancer treatment can be selected from chemotherapy, radiotherapy, surgery, immunotherapy, cell therapy, or biologic therapy.

For the purposes of any of the foregoing aspects or embodiments, the methods may further comprise repeating (d)-(h) with a second, third, fourth, fifth, sixth, seventh, eight, nineth, or tenth further sample of blood, plasma, or serum at successive time points. In some embodiments, (d)-(h) are repeated one or more times while the patient is in remission. In some embodiments, (d)-(h) are repeated one or more times while the patient is undergoing treatment for the cancer. In some embodiments, (d)-(h) are repeated one or more times coinciding with or prior to surgery; following, during, or prior to administration of chemotherapy; following, during, or prior to radiation therapy; following, during, or prior to administration of an immunotherapy; following, during, or prior to administration of a cell therapy; or following, during, or prior to administration of a biologic therapy.

For the purposes of any of the foregoing aspects or embodiments, the tumor or cancer may be selected from adrenal cancer, anal cancer, bile duct cancer, bladder cancer, bone cancer, a brain/CNS tumor, breast cancer, Castleman disease, cervical cancer, colon or rectum cancer, endometrial cancer, esophagus cancer, a Ewing tumor, eye cancer, gallbladder cancer, a gastrointestinal carcinoid tumor, a gastrointestinal stromal tumor (GIST), gestational trophoblastic disease, Hodgkin disease, Kaposi sarcoma, kidney cancer, laryngeal and hypopharyngeal cancer, leukemia, liver cancer, lung cancer, lymphoma, malignant mesothelioma, multiple myeloma, myelodysplastic Syndrome, nasal cavity or paranasal sinus cancer, nasopharyngeal cancer, neuroblastoma, oral cavity or oropharyngeal cancer, osteosarcoma, ovarian cancer, pancreatic cancer, penile cancer, a pituitary tumor, prostate cancer, retinoblastoma, rhabdomyosarcoma, salivary gland cancer, skin cancer, small intestine cancer, stomach cancer, testicular cancer, thymus cancer, thyroid cancer, uterine sarcoma, vaginal cancer, vulvar cancer, Waldenstrom macroglobulinemia, and Wilms tumor.

### BRIEF DESCRIPTION OF THE DRAWING

FIG. 1 shows allele balances observed for targets detected in a reference set of buffy coat samples. Allele balance is at 1.7%.

FIG. 2 shows variant class assignments for a cfDNA sample using expectation-maximization to fit the model. The 3-class model was run on the cfDNA capture data from good (close to truth) starting parameter values. The loop was ended at iteration 2.

FIG. 3 shows variant class assignments for a cfDNA sample using expectation-maximization to fit the model. The 3-class model was run on the cfDNA capture data from bad (implausible) starting parameter values. The loop was ended at iteration 4.

FIG. 4 shows variant class assignments for a negative control sample using expectation-maximization to fit the model. The 3-class model was run on buffy normal negative control capture data with no up-front filters on variants passed to the model. The loop was ended at iteration 2.

FIG. 5 shows a component diagram of an example computing system suitable for use in the various implementations described herein.

FIG. 6 shows a comparison of tumor fraction (TF) estimates. Tumor fraction estimate was determined using a likelihood model or two alternatives, cumulative allele fraction (AF) model or median AF model. Each point is the median value for each estimation method across 100 simulations with 150 targets. The likelihood model used a fixed dropout rate of 10% and error rates estimated from control sites. The dashed line shows identity, where estimated and expected TF are equal. The full likelihood model estimates are the most consistent with the expected TF of each sample of the three methods.

FIG. 7 shows the sensitivity of the assay for 16 target panels using either an arbitrarily low value of error rate (arbitrary error) or error rates matching sample and target-specific rates derived from control site data (per type error) at 0.005, 0.01, 0.1, or 1% tumor fraction (TF).

FIG. 8 shows the estimated tumor fraction (TF) compared to the expected TF using a 0% dropout rate or a 10% dropout rate.

### I. DEFINITIONS

As used herein, the singular forms “a,” “an,” and “the” include the plural reference unless the context clearly dictates otherwise.

Reference to “about” a value or parameter herein includes (and describes) variations that are directed to that value or parameter per se. The term “about” is used herein to mean plus or minus ten percent (10%) of a value. For example, “about 100” refers to any number between 90 and 110.

It is understood that aspects and variations of the invention described herein include “consisting” and/or “consisting essentially of” aspects and variations.

Numeric ranges are inclusive of the numbers defining the range.

Unless otherwise indicated, nucleic acids are written left to right in 5′ to 3′ orientation; amino acid sequences are written left to right in amino to carboxy orientation, respectively.

The term “mutation” herein refers to a change introduced into a reference sequence, including, but not limited to, substitutions, insertions, deletions (including truncations) relative to the reference sequence. Mutations can involve large sections of DNA (e.g., copy number variation). Mutations can involve whole chromosomes (e.g., aneuploidy). Mutations can involve small sections of DNA. Examples of mutations involving small sections of DNA include, e.g., point mutations or single nucleotide polymorphisms (SNPs), single nucleotide variant (SNV), multiple nucleotide polymorphisms, insertions (e.g., insertion of one or more nucleotides at a locus but less than the entire locus), multiple nucleotide changes, deletions (e.g., deletion of one or more nucleotides at a locus), inversions (e.g., reversal of a sequence of one or more nucleotides), an genomic rearrangements (e.g., deletions, duplications, inversions, and translocations). In some embodiments, the reference sequence is a parental sequence. In some embodiments, the reference sequence is a reference human genome, e.g., h19. In some embodiments, the reference sequence is derived from a non-cancer (or non-tumor) sequence. In some embodiments, the mutation is inherited. In some embodiments, the mutation is spontaneous or de nova. In some embodiments, the mutation is a “somatic” mutation or variant.

The term “somatic variant” or “somatic mutation” herein refers to a variant arising after conception, in non-germline DNA of an individual. Somatic variants may include single-nucleotide variants (SNVs), multi-nucleotide variants, insertions and deletions (e.g., indel variants), and genomic rearrangements for example. The terms “somatic variant” and “somatic mutation” are used interchangeably herein. In some embodiments, the terms “somatic variant” or “somatic mutation” refers to a collection of somatic variants that are specific to a patient.

The term “patient-specific panel” or “somatic variants” herein refers to a collection of sequences comprising somatic mutations that are specific to a patient, or markers that distinguish between two or more individuals. A signature panel may distinguish one sample from another.

The term “reference panel” herein refers to a collection of sequence prepared in the same way as the patient-specific panel but in a non-tumor sample. In some embodiments, the sample used to prepare the reference panel is from a healthy subject. In some embodiments, the sample is from the same patient at a time before the patient had cancer. In some embodiments, the reference panel is a known buffy coat sequence.

The term “tumor fraction” herein refers to the proportion of circulating cell-free tumor DNA (ctDNA) relative to the total amount of cell-free DNA (cfDNA). Tumor fraction may be indicative of the size of the tumor.

The term “genomic DNA” refers to DNA of a cellular genome. The genomic DNA can be cellular, i.e., contained within a cell, or it can be cell free.

The term “sample” herein refers to any substance containing or presumed to contain nucleic acid. The sample can be a biological sample obtained from a subject or patient. The nucleic acids can be RNA, DNA, e.g., genomic DNA. In some embodiments, the biological sample is a biological fluid sample. The fluid sample can be whole blood, plasma, serum, ascites, cerebrospinal fluid, sweat, urine, tears, saliva, buccal sample, cavity rinse, or organ rinse. The fluid sample can be an essentially cell-free liquid sample (e.g., plasma, serum, sweat, urine, tears, etc.). In other embodiments, the biological sample is a solid biological sample, e.g., feces or tissue biopsy, such as a tumor biopsy. In some embodiment, the sample is a tumor sample. In some embodiments, the sample is a non-tumor sample. A “sample” may include, but is not limited to, tissue, blood, plasma, saliva, urine, semen, amniotic fluid, oocytes, skin, hair, feces, cheek swabs, or pap smear lysate from an individual. In some embodiments, the sample is blood, plasma, or serum.

The term “target sequence” herein refers to a selected target polynucleotide, e.g., a sequence present in a cfDNA molecule, whose presence, amount, and/or nucleotide sequence, or changes in these, are desired to be determined. Target sequences are interrogated for the presence or absence of a somatic variant. The target polynucleotide can be a region of gene associated with a disease. In some embodiments, the region is an exon. The disease can be cancer.

The terms “anneal,” “hybridize,” or “bind,” can refer to two polynucleotide sequences, segments or strands, and can be used interchangeably and have the usual meaning in the art. Two complementary sequences (e.g., DNA and/or RNA) can anneal or hybridize by forming hydrogen bonds with complementary bases to produce a double-stranded polynucleotide or a double-stranded region of a polynucleotide.

The term “marker” or “segregating marker” refers to a moiety that is used to discriminate between two or more samples, e.g., two or more individuals or tissues. A marker may be a nucleic acid (e.g., a gene), small molecule, peptide, fatty acid, metabolite, protein, lipid, etc. A marker may be a mutation. A marker may be a synthetic nucleic acid. A marker or set of markers may define a genetic signature of an entity, e.g., an individual, relative to a second nucleic acid, e.g., a reference nucleic acid sequence.

The terms “treat,” “treatment,” and “treating” refer to the reduction or amelioration of the progression, severity, and/or duration of a proliferative disorder e.g., cancer, or the amelioration of a proliferative disorder resulting from the administration of one or more therapies.

As used herein, the term “barcode” (also termed single molecule identifier or SMI) refers to a known nucleic acid sequence that allows some feature of a polynucleotide with which the barcode is associated to be identified. In some embodiments, the feature of the polynucleotide to be identified is the sample from which the polynucleotide is derived. In some embodiments, barcodes are about or at least about 3, 4, 5, 6, 7, 8, 9, 10, 11, 12, 13, 14, 15, or more nucleotides in length. In some embodiments, barcodes are shorter than 10, 9, 8, 7, 6, 5, or 4 nucleotides in length. In some embodiments, barcodes associated with some polynucleotides are of different lengths than barcodes associated with other polynucleotides. In general, barcodes are of sufficient length and include sequences that are sufficiently different to allow the identification of samples based on barcodes with which they are associated. In some embodiments, a barcode, and the sample source with which it is associated, can be identified accurately after the mutation, insertion, or deletion of one or more nucleotides in the barcode sequence, such as the mutation, insertion, or deletion of 1, 2, 3, 4, 5, 6, 7, 8, 9, 10, or more nucleotides. In some embodiments, each barcode in a plurality of barcodes differ from every other barcode in the plurality at least three nucleotide positions, such as at least 3, 4, 5, 6, 7, 8, 9, 10, or more nucleotide positions. A plurality of barcodes may be represented in a pool of samples, each sample including polynucleotides comprising one or more barcodes that differ from the barcodes contained in the polynucleotides derived from the other samples in the pool. Samples of polynucleotides including one or more barcodes can be pooled based on the barcode sequences to which they are joined, such that all four of the nucleotide bases A, G, C, and T are approximately evenly represented at one or more positions along each barcode in the pool (such as at 1, 2, 3, 4, 5, 6, 7, 8, or more positions, or all positions of the barcode).

The term “small nucleotide polymorphism” or “SNP” refers to a single-nucleotide variant (SNV), a multi-nucleotide variant (MNV), or an indel variant about 100 base pairs or less.

The term “copy number variant,” “CNV,” or “copy number” refers to any duplication or deletion of a genomic segment. In some embodiments, the copy number is the copy number of each somatic variant in the set of somatic variants.

The term “allele balance” refers to a ratio of a variant allele to a reference allele. In some embodiments, the variant allele is a variant allele from each somatic variant in the set of somatic variants.

The term “derived from” encompasses the terms “originated from,” “obtained from,” “obtainable from,” “isolated from,” and “created from,” and generally indicates that one specified material (e.g., a biological sample) finds its origin in another specified material or individual or has features that can be described with reference to the another specified material.

The term “library” or “sequencing library” herein refers to a collection or plurality of template molecules, i.e., target DNA duplexes, which share common sequences at their 5′ ends and common sequences at their 3′ ends. Use of the term “library” to refer to a collection or plurality of template molecules should not be taken to imply that the templates making up the library are derived from a particular source, or that the “library” has a particular composition. By way of example, use of the term “library” should not be taken to imply that the individual templates within the library must be of different nucleotide sequence or that the templates must be related in terms of sequence and/or source. In general, the term “sequencing library” herein refers to DNA that is processed for sequencing, e.g., using massively parallel methods, e.g., NGS. The DNA may optionally be amplified to obtain a population of multiple copies of processed DNA, which can be sequenced by NGS.

The term “Next Generation Sequencing” or “NGS” refers to sequencing methods that allow for massively parallel sequencing of clonally amplified and of single nucleic acid molecules during which a plurality, e.g., millions, of nucleic acid fragments from a single sample or from multiple different samples are sequenced in unison. Non-limiting examples of NGS include sequencing-by-synthesis, sequencing-by-ligation, real-time sequencing, and nanopore sequencing.

The term “sequence read” or simply “read” herein refers to sequence information of a nucleic acid fragment obtained through a sequencing assay, such as a next generation sequencing (NGS) assay. In some embodiments, a sequence read refers to data representing a sequence of nucleotide bases that were measured using a clonal sequencing method. Clonal sequencing may produce sequence data representing single, or clones, or clusters of one original DNA molecule. A sequence read may also have associated quality score at each base position of the sequence indicating the probability that nucleotide has been called correctly.

The term “mapping a sequence read” herein refers to the process of determining a sequence read's location of origin in the genome sequence of a particular organism. The location of origin of sequence reads is based on similarity of nucleotide sequence of the read and the genome sequence.

The term “preferential enrichment” of DNA that corresponds to a locus, or preferential enrichment of DNA at a locus, refers to any method that results in the percentage of molecules of DNA in a post-enrichment DNA mixture that correspond to the locus being higher than the percentage of molecules of DNA in the pre-enrichment DNA mixture that correspond to the locus. The method may involve selective amplification of DNA molecules that correspond to a locus. The method may involve removing DNA molecules that do not correspond to the locus. The method may involve a combination of methods. The degree of enrichment is defined as the percentage of molecules of DNA in the post-enrichment mixture that correspond to the locus divided by the percentage of molecules of DNA in the pre-enrichment mixture that correspond to the locus. Preferential enrichment may be carried out at a plurality of loci. In some embodiments of the present disclosure, the degree of enrichment is greater than 20. In some embodiments of the present disclosure, the degree of enrichment is greater than 200. In some embodiments of the present disclosure, the degree of enrichment is greater than 2,000. When preferential enrichment is carried out at a plurality of loci, the degree of enrichment may refer to the average degree of enrichment of all of the loci in the set of loci.

The term “amplification,” with respect to nucleic acid sequences, herein refers to methods that increase the representation of a population of nucleic acid sequences in a sample. Copies of a particular target nucleic acid sequence generated in vitro in an amplification reaction are called “amplicons” or “amplification products”. Amplification may be exponential or linear. A target nucleic acid may be DNA (such as, for example, genomic DNA, cfDNA, ctDNA, and cDNA) or RNA. While the exemplary methods described hereinafter relate to amplification using polymerase chain reaction (PCR), numerous other methods such as isothermal methods, rolling circle methods, etc., are available to the skilled artisan. The skilled artisan will understand that these other methods may be used either in place of, or together with, PCR methods. See, e.g., Saiki, “Amplification of Genomic DNA” in PCR PROTOCOLS, Innis et al., Eds., Academic Press, San Diego, CA 1990, pp 13-20; Wharam, et al., Nucleic Acids Res. 29(11): E54-E54 (2001).

The term “selective amplification” herein refers to a method that increases the number of copies of a particular molecule of DNA, or molecules of DNA that correspond to a particular region of DNA. It may also refer to a method that increases the number of copies of a particular targeted molecule of DNA, or targeted region of DNA more than it increases non-targeted molecules or regions of DNA. Selective amplification may be a method of preferential enrichment.

The term “direct amplification” herein refers to a nucleic acid amplification reaction in which the target nucleic acid is amplified from the sample without prior purification, extraction, or concentration.

The term “amplification mixture” herein refers to a mixture of reagents that are used in a nucleic acid amplification reaction, but does not contain primers or sample. An amplification mixture comprises a buffer, dNTPs, and a DNA polymerase. An amplification mixture may further comprise at least one of MgCl₂, KCl, nonionic and ionic detergents (including cationic detergents). In general, amplification methods disclosed herein with include an amplification mixture. The term “amplification master mix” refers to an amplification mixture, primers, and/or probes for amplifying one or more target nucleic acids, but does not contain the sample to be amplified. The term “reaction-sample mixture” herein refers to a mixture containing amplification master mix and a sample.

The term “multiplex PCR” herein refers to the simultaneous generation of two or more PCR products or amplicons within the same reaction vessel. Similarly, a “2-plex PCR” refers to the simultaneous generation of two PCR products or amplicons within the same reaction vessel. Each PCR product is primed using a distinct primer pair. A multiplex reaction may further include specific probes for each product that are labeled with different detectable moieties.

The term “universal priming sequence” refers to a DNA sequence that may be appended to a population of target DNA molecules, for example by ligation, PCR, or ligation mediated PCR. Once added to the population of target molecules, primers specific to the universal priming sequences can be used to amplify the target population using a single pair of amplification primers. Universal priming sequences are typically not related to the target sequences.

The term “universal adapters” or “ligation adaptors” or “library tags” are DNA molecules containing a universal priming sequence that can be covalently linked to the 5-prime and 3-prime end of a population of target double stranded DNA molecules. The addition of the adapters provides universal priming sequences to the 5-prime and 3-prime end of the target population from which PCR amplification can take place, amplifying all molecules from the target population, using a single pair of amplification primers.

The term “targeting” herein refers to a method used to selectively amplify or otherwise preferentially enrich those molecules of DNA that correspond to a set of loci, in a mixture of DNA.

The term “primer” herein refers to an oligonucleotide, whether occurring naturally or produced synthetically, which is capable of acting as a point of initiation of nucleic acid synthesis when placed under conditions in which synthesis of a primer extension product which is complementary to a nucleic acid strand is induced, e.g., in the presence of four different nucleotide triphosphates and a polymerase enzyme, e.g., a thermostable enzyme, in an appropriate buffer (“buffer” includes pH, ionic strength, cofactors, etc.) and at a suitable temperature. The primer is preferably single-stranded for maximum efficiency in amplification, but may alternatively be double-stranded. If double-stranded, the primer is first treated to separate its strands before being used to prepare extension products. Preferably, the primer is an oligodeoxyribonucleotide. The primer must be sufficiently long to prime the synthesis of extension products in the presence of the polymerase, e.g., thermostable polymerase enzyme. The exact lengths of a primer will depend on many factors, including temperature, source of primer and use of the method. For example, depending on the complexity of the target sequence, the oligonucleotide primer typically contains 15-25 nucleotides, although it may contain more or few nucleotides. Short primer molecules generally require colder temperatures to form sufficiently stable hybrid complexes with template.

A “hybrid capture probe” herein refers to any nucleic acid sequence, possibly modified, that is generated by various methods such as PCR or direct synthesis and intended to be complementary to one strand of a specific target DNA sequence in a sample. The exogenous hybrid capture probes may be added to a prepared sample and hybridized through a denature-reannealing process to form duplexes of exogenous-endogenous fragments. These duplexes may then be physically separated from the sample by various means.

A “spacer” may consist of a repeated single nucleotide (e.g., 1, 2, 3, 4, 5, 6, 7, 8, 9, 10, or more of the same nucleotide in a row), or a sequence of 2, 3, 4, 5, 6, 7, 8, 9, 10, or more nucleotides repeated 1, 2, 3, 4, 5, 6, 7, 8, 9, 10, or more times. A spacer may comprise or consist of a specific sequence, such as a sequence that does not hybridize to any target sequence in a sample. A spacer may comprise or consist of a sequence of randomly selected nucleotides.

The phrases “substantially similar” and “substantially identical” in the context of at least two nucleic acids typically means that a polynucleotide includes a sequence that has at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or even 99.5% sequence identity, in comparison with a reference (e.g., wild-type) polynucleotide or polypeptide. Sequence identity may be determined using known programs such as BLAST, ALIGN, and CLUSTAL using standard parameters. (See, e.g., Altshul et al. (1990) J. Mol. Biol. 215:403-410; Henikoff et al. (1989) Proc. Natl. Acad. Sci. 89:10915; Karin et al. (1993) Proc. Natl. Acad. Sci. 90:5873; and Higgins et al. (1988) Gene 73:237). Software for performing BLAST analyses is publicly available through the National Center for Biotechnology Information. Also, databases may be searched using FASTA (Person et al. (1988) Proc. Natl. Acad. Sci. 85:2444-2448.) In some embodiments, substantially identical nucleic acid molecules hybridize to each other under stringent conditions (e.g., within a range of medium to high stringency).

The term “tag” refers to a detectable moiety that may be one or more atom(s) or molecule(s), or a collection of atoms and molecules. A tag may provide an optical, fluorescent, electrochemical, magnetic, or electrostatic (e.g., inductive, capacitive) signature.

The term “tagged nucleotide” herein refers to a nucleotide that includes a tag (or tag species) that is coupled to any location of the nucleotide including, but not limited to a phosphate (e.g., terminal phosphate), sugar or nitrogenous base moiety of the nucleotide. Tags may be one or more atom(s) or molecule(s), or a collection of atoms and molecules. A tag may provide an optical, electrochemical, magnetic, or electrostatic (e.g., inductive, capacitive) signature.

As used herein, the term “target polynucleotide” refers to a nucleic acid molecule or polynucleotide in a population of nucleic acid molecules having a target sequence to which one or more oligonucleotides are designed to hybridize. “Target polynucleotide” may be used to refer to a double-stranded nucleic acid molecule that includes a target sequence on one or both strands, or a single-stranded nucleic acid molecule including a target sequence, and may be derived from any source of or process for isolating or generating nucleic acid molecules. A target polynucleotide may include one or more (e.g., 1, 2, 3, 4, 5, 6, 7, 8, 9, 10, or more) target sequences, which may be the same or different. In general, different target polynucleotides include different sequences, such as one or more different nucleotides or one or more different target sequences.

The term “template DNA molecule” herein refers to a strand of a nucleic acid from which a complementary nucleic acid strand is synthesized by a DNA polymerase, for example, in a primer extension reaction.

A “portion adjacent to a region of interest” refers to a sequence that is immediately proximal to a region of interest. Reference to a “portion of or adjacent to a region of interest” refers to a sequence that 1) is entirely within the region of interest, 2) is entirely outside but immediately proximal to the region of interest, or 3) includes a contiguous sequence from within and immediately proximal to the region of interest. Reference to a “sequence that is substantially complementary to a portion of or adjacent to a region of interest” refers to 1) a sequence that is substantially complementary to a sequence entirely within the region of interest, 2) a sequence substantially complementary to a sequence entirely outside but immediately proximal to the region of interest, or 3) a sequence that is substantially complementary to a contiguous sequence from with and immediately proximal to the region of interest.

A “control site” as used herein refers to a corresponding site for each somatic variant located, for example, anywhere within the sequence library or in any sequence reads, within about 160 bases, with about 120 bases, or within 20 bases of the somatic variant on the DNA fragments (e.g., 20, 19, 18, 17, 16, 15, 14, 13, 12, 11, 10, 9, 8, 7, 6, 5, 4, 3, 2, or 1). In some embodiments, the corresponding site is within about 1 base of the somatic variant. In some embodiments, the corresponding site is within about 2 bases of the somatic variant. In some embodiments, the corresponding site is within about 3 bases of the somatic variant. In some embodiments, the corresponding site comprises a reference base that is the same as the base of the somatic variant.

“Noisy Genetic Data” herein refers to genetic data with any of the following: allele dropouts, uncertain base pair measurements, incorrect base pair measurements, missing base pair measurements, uncertain measurements of insertions or deletions, uncertain measurements of chromosome segment copy numbers, spurious signals, missing measurements, other errors, or combinations thereof.

“Confidence” herein refers to the statistical likelihood that the called SNP, SNV, variant, copy number, etc. correctly represents the real genetic state of the individual.

### II. MINIMAL RESIDUAL DISEASE DETECTION

The goal of a minimum residual disease (MRD) assay is to detect and/or quantify circulating tumor DNA (ctDNA) so researchers and clinicians can detect recurrence early and monitor the progress of the disease through treatment. In general, an MRD assay will rely on a patient-specific and tumor-specific panel (i.e., a “signature panel” or “somatic variants”) for assessing the presence of ctDNA in a patient sample. The signature panel can be prepared with the general steps of (1) profiling a tumor or cancer sample from a patient, and (2) identifying a somatic mutations to target, and, at one or more later time points, (3) taking a subsequent sample from the patient, (4) enriching cell-free DNA (cfDNA) for the target somatic mutation sites, and (5) determining or estimating the ctDNA content of the cfDNA given the tumor profile and sequencing data.

More specifically, preparing the patient-specific and tumor-specific panel (i.e., a “signature panel” or “somatic variants”) may comprise, for example, (a) obtaining a tumor sample and a non-tumor sample from a cancer patient; (b) sequencing DNA (e.g., genomic DNA) from the tumor sample and sequencing DNA (e.g., cfDNA) from the non-tumor sample, thereby obtaining sequences DNA or sequence reads from the tumor sample and the non-tumor sample; and (c) comparing the sequences of the tumor sample and the non-tumor sample to determine any tumor-specific somatic mutations that are present in the sequences of DNA from the tumor sample but not present in the sequences of DNA from the non-tumor sample. Sequencing of the DNA form the tumor sample and non-tumor sample may comprise whole genome sequencing or various types of targeted sequencing, such as whole exome sequencing.

This comparison of the tumor and non-tumor sequences can be performed by, for example, aligning the sequences of DNA (e.g., genomic DNA) from the tumor sample to a reference human genome that is not from the patient and aligning the sequences of DNA (e.g., cfDNA) from the non-tumor sample to the reference genome that is not from the patient. The reference genome can be, for example, a publicly available human genome assembly, such as hg18, hg19, GRCh38.p14, GRCh37.p13, or other assemblies from the Genome Reference Consortium. Alternatively, the comparison of the tumor and non-tumor sequences can be performed by, for example, aligning the sequences of DNA (e.g., genomic DNA) from the tumor sample to sequences of DNA (e.g., cfDNA) from the non-tumor sample. With either approach, the skilled artisan is able to detect and identify tumor-specific somatic mutations that are present in the tumor sample but not in the non-tumor sample.

The tumor sample may be a solid tumor sample, such as a biopsy or other tissue sample, or a liquid sample or a fluid sample, such as blood (in the case of a hematological cancer) or specific fractions of blood. The non-tumor sample may be tissue-matched with the tumor sample or it may be from a different tissue. For example, the non-tumor sample may be selected from a healthy (i.e., non-cancerous or non-tumor) tissue sample, blood or specific fractions of blood such as buffy coat, leukocytes, fibroblast, or any other biological sample comprising cfDNA or genomic DNA. In some embodiment, the tumor sample comprises a tumor biopsy or fluid sample. In some embodiments, the fluid sample is selected from blood, blood plasma, blood serum, urine, saliva, and cerebral spinal fluid (CSF). In some embodiment, the non-tumor sample comprises a tissue sample matched to a tissue of origin of the tumor sample. In some embodiments, the non-tumor sample comprises a fluid sample selected from a buffy coat sample, blood, blood plasma, blood serum, urine, saliva, and cerebral spinal fluid (CSF).

Once a patient-specific and tumor-specific panel (i.e., a “signature panel” or “somatic variants”) has been established, such a signature panel can be used to enrich ctDNA in subsequent samples taken from the cancer patient. The subsequent samples may be taken from a patient at various time points during the course of treatment or during a period of remission. For example, after a surgical removal of a tumor, the tumor may be profiled as described herein to determine tumor-specific somatic mutations, and at one or more subsequent time points a subsequent sample may be taken from the subject to search for the presence of any ctDNA comprising any one of the identified tumor-specific somatic mutations. The detection or presence of ctDNA comprising a tumor-specific somatic mutation may be indicative of cancer recurrence. Additionally or alternatively, similar assessment can be performed throughout the course of a patient's treatment (e.g., with chemotherapy, radiation, immunotherapy, cell therapy, biologic therapy, etc.) to detect or quantify ctDNA and determine whether the amount of ctDNA is increasing or decreasing, as this may be indicative of responsiveness to the therapy. Accordingly, assessment of a subsequent sample may be repeated 1, 2, 3, 4, 5, 6, 7, 8, 9, or 10 or more times throughout the course of a patient's remission or treatment. The assessment of a subsequent sample may be repeated monthly, every other month, once every three months, once every four months, once every five months, once every six months, once every seven months, once every eight months, once every nine months, once every ten months, once every eleven months, or annually.

The type of sample used for the one or more subsequent samples is generally a blood sample, a plasma sample, or a serum sample, but any biological sample that contains cfDNA and potential contains ctDNA would be acceptable. In some embodiments, the one or more subsequent samples are cell-free samples.

Enrichment of ctDNA (e.g., fragments that include a target sequence corresponding to a tumor-specific somatic mutation or variant) in the one or more subsequent samples can be performed by methods including, but not limited to, hybrid capture-based enrichment, PCR-target enrichment, or on-sequencer enrichment. Briefly, enrichment may comprise extracting cfDNA from a subsequent sample taken from the cancer patient and contacting the extracted cfDNA with a plurality of oligonucleotides (i.e., oligonucleotide probes), wherein each oligonucleotide in the plurality of oligonucleotides comprises a nucleic acid sequence that is capable of hybridizing to a cfDNA fragment comprising one of the tumor-specific somatic mutation sequences identified by comparing the sequences of the patients tumor DNA and non-tumor DNA. In some embodiments, the nucleic acid sequence is capable of hybridizing 1 or more nucleotide bases upstream or downstream of the tumor-specific somatic mutation sequences. Thus, enrichment may utilize a set of oligonucleotide probes to selectively enrich ctDNA that may be in the subsequent sample by binding to previously identified tumor-specific somatic mutation sequences.

A signature panel or somatic variants may comprise 10-5000 tumor-specific somatic mutations. For example, a signature panel may comprise 10-4000, 10-3000, 10-2500, 10-2000, 10-1500, 10-1000, 10-950, 10-900, 10-850, 10-800, 10-750, 10-700, 10-650, 10-600, 10-550, 10-500, 50-5000, 50-4000, 50-3000, 50-2500, 50-2000, 50-1500, 50-1000, 50-950, 50-900, 50-850, 50-800, 50-750, 50-700, 50-650, 50-600, 50-550, 50-500, 100-5000, 100-4000, 100-3000, 100-2500, 100-2000, 100-1500, 100-1000, 100-950, 100-900, 100-850, 100-800, 100-750, 100-700, 100-650, 100-600, 100-550, 100-500, 200-5000, 200-4000, 200-3000, 200-2500, 200-2000, 200-1500, 200-1000, 200-950, 200-900, 200-850, 200-800, 200-750, 200-700, 200-650, 200-600, 200-550, 200-500, 300-5000, 300-4000, 300-3000, 300-2500, 300-2000, 300-1500, 300-1000, 300-950, 300-900, 300-850, 300-800, 300-750, 300-700, 300-650, 300-600, 300-550, 300-500, 400-5000, 400-4000, 400-3000, 400-2500, 400-2000, 400-1500, 400-1000, 400-950, 400-900, 400-850, 400-800, 400-750, 400-700, 400-650, 400-600, 400-550, 400-500, 500-5000, 500-4000, 500-3000, 500-2500, 500-2000, 500-1500, 500-1000, 500-950, 500-900, 500-850, 500-800, 500-750, 500-700, 500-650, 500-600, or 500-550 tumor-specific somatic mutations. In some embodiments, a signature panel or somatic variants may comprise or consist of about 10, about 20, about 30, about 40, about 50, about 75, about 100, about 150, about 200, about 250, about 300, about 350, about 400, about 450, about 500, about 550, about 600, about 650, about 700, about 750, about 800, about 850, about 900, about 950, about 1000, about 1100, about 1150, about 1200, about 1250, about 1300, about 1350, about 1400, about 1450, about 1500, about 1550, about 1600, about 1650, about 1700, about 1750, about 1800, about 1850, about 1900, about 1950, or about 2000 or more tumor-specific somatic mutations. In some embodiments, a signature panel or somatic variants may comprise at least 10, at least 20, at least 30, at least 40, at least 50, at least 75, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1100, at least 1150, at least 1200, at least 1250, at least 1300, at least 1350, at least 1400, at least 1450, at least 1500, at least 1550, at least 1600, at least 1650, at least 1700, at least 1750, at least 1800, at least 1850, at least 1900, at least 1950, or at least 2000 tumor-specific somatic mutations. The tumor-specific somatic mutations may be in introns, exons, or a combination thereof. In some embodiments, the tumor-specific mutations may be one or more somatic mutations selected from SNVs, insertions, deletions and translocations.

After enrichment or concurrently with enrichment of ctDNA (e.g., fragments that include a target sequence corresponding to a tumor-specific somatic mutation or variant), the enriched DNA is sequenced. This sequencing may be performed by, for example Next Generation Sequencing (NGS). Deep sequencing may allow for more sensitive detection, and so the depth of the sequencing may be at least 50×, at least 100×, at least 150×, at least 200×, at least 250×, at least 300×, at least 350×, at least 400×, at least 450×, at least 500×, at least 550×, at least 600×, at least 650×, at least 700×, at least 750×, at least 800×, at least 850×, at least 900×, at least 950×, or at least 1000×. In other words, the depth of the sequencing may be about 50×, about 100×, about 150×, about 200×, about 250×, about 300×, about 350×, about 400×, about 450×, about 500×, about 550×, about 600×, about 650×, about 700×, about 750×, about 800×, about 850×, about 900×, about 950×, or about 1000×. The detection sensitivity of the disclosed methods may be about 20 to about 50 ctDNA fragments comprising one or more of the set of somatic mutations in the fluid sample per a total background of about 500,000 cfDNA fragments.

The disclosed methods may be used for tracking and assessing recurrence in any cancer patient. For example, the cancer patient may have a cancer selected from, but not limited to, adrenal cancer, anal cancer, bile duct cancer, bladder cancer, bone cancer, a brain/CNS tumor, breast cancer, Castleman disease, cervical cancer, colon or rectum cancer, endometrial cancer, esophagus cancer, a Ewing tumor, eye cancer, gallbladder cancer, a gastrointestinal carcinoid tumor, a gastrointestinal stromal tumor (GIST), gestational trophoblastic disease, Hodgkin disease, Kaposi sarcoma, kidney cancer, laryngeal and hypopharyngeal cancer, leukemia, liver cancer, lung cancer, lymphoma, malignant mesothelioma, multiple myeloma, myelodysplastic Syndrome, nasal cavity or paranasal sinus cancer, nasopharyngeal cancer, neuroblastoma, oral cavity or oropharyngeal cancer, osteosarcoma, ovarian cancer, pancreatic cancer, penile cancer, a pituitary tumor, prostate cancer, retinoblastoma, rhabdomyosarcoma, salivary gland cancer, skin cancer, small intestine cancer, stomach cancer, testicular cancer, thymus cancer, thyroid cancer, uterine sarcoma, vaginal cancer, vulvar cancer, Waldenstrom macroglobulinemia, and Wilms tumor. In some embodiments, the cancer may be a blood borne or hematological cancer such as leukemia or lymphoma.

The disclosed MRD assay, specifically the obtaining and testing of subsequent samples from a cancer patient, may be repeated one or more times following completion of a cancer treatment; one or more times while the cancer patient is in remission; one or more times coinciding with or prior to surgery; following, during, or prior to administration of chemotherapy; following, during, or prior to radiation therapy; following, during, or prior to immunotherapy; or following, during, or prior to cell therapy; or following, during, or prior to administration of a biologic therapy. The disclosed MRD assay may also be repeated at times prior to, coinciding with, and/or following an imaging test, such as a PET scan, a PET/CT scan, an MRI, or an X-ray.

The disclosed methods allow for detecting ctDNA or determining the tumor fraction from a biological sample from a patient that has, previously had, or is suspected of having cancer. As described in further detail below, the methods can be represented by two phases. In a first phase, or enrollment phase, somatic mutations or variants that are specific to a patient are identified. A panel of probes (e.g., capture probes) is then generated that are specific to the subset panel of somatic mutations or variants, which can be used to enrich a sample before sequencing.

Specific aspects of MRD processes are discussed on more detail below.

### III. PHASE I—SIGNATURE PANEL OF MARKERS/MUTATIONS AND CAPTURE PROBES

a. DNA Library Preparation

In some embodiments of the methods disclosed herein, a DNA library is obtained or prepared from cfDNA obtained from a patient, e.g., a cancer patient. In some embodiments, a DNA library is obtained or prepared from the genome of the patient. In some embodiments, the DNA has been previously sequenced and mutations or variants identified.

When producing a DNA library from genomic DNA, the genomic DNA can be fragmented, for example by using a hydrodynamic shear or other mechanical force, or fragmented by chemical or enzymatic digestion, such as restriction digesting. This fragmentation process allows the DNA molecules present in the genome to be sufficiently short for analysis, such as sequencing or digital PCR. cfDNA, however, is generally sufficiently short such that no fragmentation is necessary. cfDNA originates from genomic DNA. A portion of the cfDNA obtained from a plasma sample of a cancer patient may originate from cancer cells (i.e., circulating tumor DNA or ctDNA) and a portion of the cfDNA may originate from non-cancer cells.

In some embodiments, the DNA molecules are subjected to additional modification, resulting in the attachment of oligonucleotides to the DNA molecules. The oligonucleotides can comprise an adapter sequence or a molecular barcode (or both). In some embodiments, the adapter sequence is common to all oligonucleotides in a plurality of oligonucleotides that are used to form the DNA library. In some embodiments, the molecular barcodes are unique or have low redundancy. By way of example, the oligonucleotide can be attached to the DNA molecules by ligation. Direct attachment of the oligonucleotides to the DNA molecules in the DNA library can be used, for example, when enrichment occurs in a downstream process. For example, in some embodiments, a DNA library is prepared by direct attachment of an oligonucleotide comprising a molecular barcode and an adapter sequence, followed by enrichment (for example, by hybridization) of DNA molecules comprising a region of interest or a portion of a region of interest.

In some embodiments, library preparation and enrichment occurs simultaneously. For example, in some embodiments, DNA molecules comprising a region of interest or a portion thereof are preferentially amplified. This can be done, for example, by combining the cfDNA (or genomic DNA), with oligonucleotides comprising a target-specific sequence, an adapter sequence, and a molecular barcode, and amplifying the DNA molecules. As before, in some embodiments, the adapter sequence is common to all oligonucleotides in a plurality of oligonucleotides, and the molecular barcode is unique or of low redundancy. The target-specific sequence is unique to the targeted region of interest or portion thereof. Thus, PCR amplification selectively amplifies the DNA molecules comprising the region of interest or portion thereof.

When the methods include the use of tags or molecular barcodes, the tag or molecular barcode may also be ligated to the fragments or included within the ligated adapter sequences. The independent attachment of the tag or molecular barcode, as opposed to incorporating the tag or molecular barcode, may vary with the enrichment method. For example, when using hybrid capture-based target enrichment the adapter can include the molecular barcode, when using PCR-targeted enrichment target-specific primer pairs and overhangs are used that will incorporate the sequencing adapters and sample-specific and molecular barcodes, and when using on-sequencer enrichment the adapter may be separately ligated from the tag or molecular barcode.

b. Panel of Mutations/Markers

In some embodiments, sequencing of the nucleic acid from the sample is performed using whole genome sequencing (WGS). In some embodiments, targeted sequencing is performed and may be either DNA or RNA sequencing. The targeted sequencing may be to a subset of the whole genome. In some embodiments the targeted sequencing is to introns, exons, intergenic regions, non-coding sequences or a combination thereof. In other embodiments, targeted whole exome sequencing (WES) of the DNA from the sample is performed. The DNA is sequenced using a next generation sequencing platform (NGS), which is massively parallel sequencing. NGS technologies provide high throughput sequence information, and provide digital quantitative information, in that each sequence read that aligns to the sequence of interest is countable. In certain embodiments, clonally amplified DNA templates or single DNA molecules are sequenced in a massively parallel fashion within a flow cell. In addition to high-throughput sequence information, NGS provides quantitative information, in that each sequence read is countable and represents an individual clonal DNA template or a single DNA molecule. The sequencing technologies of NGS include pyrosequencing, sequencing-by-synthesis with reversible dye terminators, sequencing by oligonucleotide probe ligation and ion semiconductor sequencing. DNA from individual samples can be sequenced individually (i.e., singleplex sequencing) or DNA from multiple samples can be pooled and sequenced as indexed genomic molecules (i.e., multiplex sequencing) on a single sequencing run, to generate up to several hundred million reads of DNA sequences. Commercially available platforms include, e.g., platforms for sequencing-by-synthesis, ion semiconductor sequencing, pyrosequencing, reversible dye terminator sequencing, sequencing by ligation, single-molecule sequencing, sequencing by hybridization, and nanopore sequencing. Platforms for sequencing by synthesis are available from, e.g., Illumina, 454 Life Sciences, Helicos Biosciences, and Qiagen. Illumina platforms can include, e.g., Illumina's Solexa platform, Illumina's Genome Analyzer. Life Science platforms include, e.g., the GS Flex and GS Junior, and are described in U.S. Pat. No. 7,323,305. Platforms from Helicos Biosciences include the True Single Molecule Sequencing platform. Ion Torrent, an alternative NGS system, is available from ThermoScientific and is a semiconductor based technology that detects hydrogen ions that are released during polymerization of nucleic acids. Any detection method that allows for the detection of segregatable markers may be used with the assay provided for herein.

In some embodiments, whole genome sequencing (WGS) of the tumor and normal DNA is performed.

In other embodiments, Whole Exome Sequencing (WES) of the tumor and normal DNA is performed. WES comprises selecting DNA sequences that encode proteins, and sequencing that DNA using any high throughput DNA sequencing technology. Methods that can be used to target exome DNA include the use of polymerase chain reaction (PCR), molecular inversion probes (MIP), hybrid capture, and in-solution capture. The utility of targeted genome approaches is well established, and commercially available methods for WES include the Roche NimbleGen Capture Array (Roche NimbleGen Inc., Madison, WI), Agilent SureSelect (Agilent Technologies, Santa Clara, CA), and RainDance Technologies emulsion PCR (RainDance Technologies, Lexington, MA), IDT xGen® Exome Research Panel and others.

Sequence reads may comprise about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, about 500 bp, or more than 500 bp.

In some embodiments of the methods described herein, the somatic mutations identified will be analyzed and filtered to generate a subset panel of markers. For example, the subset panel of markers may comprise one or more types of somatic mutation, including but not limited to single-nucleotide variants (SNVs) multi-nucleotide variants, insertions and deletions (e.g., indel variants), and genomic rearrangements. In some embodiments, the subset panel will only include somatic mutations that comprise multiple changes compared to the normal sample, i.e., the subset panel will not include any SNVs. In some embodiments, the subset panel of somatic mutations can include greater than 50, up to 100, up to 200, up to 300, up to 400, up to 500, up to 600, up to 700, up to 800, up to 900, up to 1,000, up to 1,500, up to 2,000, up to 2,500, up to 3,000, up to 4,000, up to 5,000, up to 6,000, up to 7,000, up to 8,000, up to 9,000, up to 10,000, up to 11,000, up to 12,000, up to 13,000, up to 14,000, up to 15,000, or more than 15,000 mutations, which may comprise MNVs, small indels, genomic rearrangements, or combinations thereof. In other embodiments, the subset panel includes between 50 and 15,000 mutations, between 100 and 15,000 mutations, between 500 and 13,000 mutations, between 1,000 and 10,000 mutations, between 2,000 and 8,000 mutations, or between 4,000 and 6,000 mutations.

c. Probes

The somatic variants or subset panel may be represented by a set of oligonucleotide probes (e.g., capture probes) each designed to at least partially hybridize to a target sequence that has been identified to comprise a mutation or variant identified in the tumor sample from the patient or in the parental sequence.

For the purposes of the disclosed methods, probes that are used to identify fragments of interest (i.e., fragments comprising a somatic mutation or variant) can include, but are not limited to capture probes, primers (for the purposes of PCR-based enrichment), or any other suitable type of nucleic acid probe.

In some embodiments, the panel comprises capture probes comprising the somatic variants identified in the patient's tumor. In some embodiments, each capture probe is designed to selectively hybridize to a target sequence. The capture probe can be at least 70%, 75%, 80%, 90%, 95%, or more than 95% complementary to a target sequence. In some embodiments, the capture probe is 100% complementary to a target sequence. In some embodiments the capture probes are DNA probes. In other embodiments, the capture probes can be RNA.

The capture probe generally is sufficiently long to encompass the sequence of a somatic variant, or corresponding normal sequence comprised in the genomic sequence targeted by the capture probe. In some embodiments, the capture probe encompasses any corresponding control site. In some embodiments, at least two capture probes are utilized, whereby a first capture probe encompasses the sequence the somatic variant and a second capture probe encompasses the corresponding control site. The length and composition of a capture probe can depend on many factors including temperature of the annealing reaction, source and base composition of the oligonucleotide, and the estimated ratio of probe to genomic target sequence. Additionally, the length of the capture probe is dependent on the length of the target sequence it is designed to capture. The method provided utilizes cfDNA including circulating tumor DNA (ctDNA) as the source of the target sequences that are to be captured. Accordingly, as cfDNA is highly fragmented to an average of about 170 bp, the capture probe can be, for example, between 100 and 300 bp, between 150 and 250 bp, or between 175 and 200 bp. Currently, methods known in the art describe probes that are typically longer than 120 bases. In a current embodiment, if the allele is one or a few bases then the capture probes may be less than about 110 bases, less than about 100 bases, less than about 90 bases, less than about 80 bases, less than about 70 bases, less than about 60 bases, less than about 50 bases, less than about 40 bases, less than about 30 bases, and less than about 25 bases, and this is sufficient to ensure equal enrichment from all alleles. When the mixture of DNA that is to be enriched using the hybrid capture technology is a mixture comprising cfDNA isolated from blood the average length of DNA is quite short, typically less than 200 bases. The use of shorter probes results in a greater chance that the hybrid capture probes will capture desired DNA fragments. Larger variations may require longer probes. For the purposes of the present disclosure, the variations of interest are more than one base in length. In some embodiments, targeted regions in the genome can be preferentially enriched using hybrid capture probes wherein the hybrid capture probes are shorter than 90 bases, and can be less than 80 bases, less than 70 bases, less than 60 bases, less than 50 bases, less than 40 bases, less than 30 bases, or less than 25 bases. In some embodiments, to increase the chance that the desired allele is sequenced, the length of the probe that is designed to hybridize to the regions flanking the polymorphic allele location can be decreased from above 90 bases, to about 80 bases, or to about 70 bases, or to about 60 bases, or to about 50 bases, or to about 40 bases, or to about 30 bases, or to about 25 bases.

Hybrid capture probes can be designed such that the region of the capture probe with DNA that is complementary to the DNA found in regions flanking the polymorphic allele is not immediately adjacent to the polymorphic site. Instead, the capture probe can be designed such that the region of the capture probe that is designed to hybridize to the DNA flanking the polymorphic site of the target is separated from the portion of the capture probe that will be in van der Waals contact with the polymorphic site by a small distance that is equivalent in length to one or a small number of bases. In an embodiment, the hybrid capture probe is designed to hybridize to a region that is flanking the polymorphic allele but does not cross it; this may be termed a flanking capture probe. The length of the flanking capture probe may be less than about 120 bases, less than about 110 bases, less than about 100 bases, less than about 90 bases, and can be less than about 80 bases, less than about 70 bases, less than about 60 bases, less than about 50 bases, less than about 40 bases, less than about 30 bases, or less than about 25 bases. The region of the genome that is targeted by the flanking capture probe may be separated by the polymorphic locus by 1, 2, 3, 4, 5, 6, 7, 8, 9, 10, 11-20, or more than 20 base pairs.

For small insertions or deletions, one or more probes that overlap the mutation may be sufficient to capture and sequence fragments comprising the mutation. Hybridization may be less efficient between the probe-limiting capture efficiency, typically designed to the reference genome sequence. To ensure capture of fragments comprising the mutation one could design two probes, one matching the normal allele and one matching the mutant allele. A longer probe may enhance hybridization. Multiple overlapping probes may enhance capture. Finally, placing a probe immediately adjacent to, but not overlapping, the mutation may permit relatively similar capture efficiency of the normal and mutant alleles.

For Short Tandem Repeats (STRs), a probe overlapping these highly variable sites is unlikely to capture the fragment well. To enhance capture a probe could be placed adjacent to, but not overlapping the variable site. The fragment could then be sequenced as normal to reveal the length and composition of the STR.

For large deletions, a series of overlapping probes, a common approach currently used in exon capture systems may work. However, with this approach it may be difficult to determine whether or not an individual is heterozygous. According to the method provided, custom probes are designed to ensure capture of the unique set of somatic mutations identified in the patient's tumor.

Capture probes can be modified to comprise purification moieties that serve to isolate the capture duplex from the unhybridized, untargeted cfDNA sequences by binding to a purification moiety binding partner. Suitable binding pairs for use in the invention include, but are not limited to, antigens/antibodies (for example, digoxigenin/antidigoxigenin, dinitrophenyl (DNP)/anti-DNP, dansyl-X-antidansyl, Fluorescein/anti-fluorescein, lucifer yellow/anti-lucifer yellow, and rhodamine anti-rhodamine); biotin/avidin (or biotin/streptavidin); calmodulin binding protein (CBP)/calmodulin; hormone/hormone receptor; lectin/carbohydrate; peptide/cell membrane receptor; protein A/antibody; hapten/antihapten; enzyme/cofactor; and enzyme/substrate. Other suitable binding pairs include polypeptides such as the FLAG-peptide (Hopp et al., BioTechnology, 6:1204-1210 (1988)); the KT3 epitope peptide (Martin et al., Science, 255:192-194 (1992)); tubulin epitope peptide (Skinner et al., J. Biol. Chem., 266:15163-15166 (1991)); and the T7 gene 10 protein peptide tag (Lutz-Freyermuth et al., Proc. Natl. Acad. Sci. USA, 87:6393-6397 (1990)) and the antibodies each thereto. Further non-limiting examples of binding partners include agonists and antagonists for cell membrane receptors, toxins and venoms, viral epitopes, hormones such as steroids, hormone receptors, peptides, enzymes and other catalytic polypeptides, enzyme substrates, cofactors, drugs including small organic molecule drugs, opiates, opiate receptors, lectins, sugars, saccharides including polysaccharides, proteins, and antibodies including monoclonal antibodies and synthetic antibody fragments, cells, cell membranes and moieties therein including cell membrane receptors, and organelles. In some embodiments, the first binding partner is a reactive moiety, and the second binding partner is a reactive surface that reacts with the reactive moiety, such as described herein with respect to other aspects of the invention. In some embodiments, the oligonucleotide primers are attached to the solid surface prior to initiating the extension reaction. Methods for the addition of binding partners to capture oligonucleotide probes are known in the art, and include addition during (such as by using a modified nucleotide comprising the binding partner) or after synthesis. Additionally, the capture probes can be tethered to a solid surface, e.g., a magnetic bead, which facilitates the isolation of captured sequences.

### IV. PHASE II-DETECTION AND MONITORING TUMORS BY ANALYZING CFDNA

a. Targeted Enrichment of a Region of Interest

The disclosed methods generally comprise enriching a target sequence in a region of interest. Examples of enrichment techniques include, but are not limited to, hybrid capture, selective circularization (also referred to as molecular inversion probes (MIP)), and PCR amplification of targeted regions of interest. Hybrid capture methods are based on the selective hybridization of the target genomic regions to user-designed oligonucleotides. The hybridization can be to oligonucleotides immobilized on high or low density microarrays (on-array capture), or solution-phase hybridization to oligonucleotides modified with a ligand (e.g., biotin) which can subsequently be immobilized to a solid surface, such as a bead (in-solution capture). Molecular inversion probe (MIP)-based method relies on construction of numerous single-stranded linear oligonucleotide probes, consisting of a common linker flanked by target-specific sequences. Upon annealing to a target sequence, the probe gap region is filled via polymerization and ligation, resulting in a circularized probe. The circularized probes are then released and amplified using primers directed at the common linker region. PCR-based methods employ highly parallel PCR amplification, where each target sequence in the sample has a corresponding pair of unique, sequence-specific primers. In some embodiments, enrichment of a target sequence occurs at the time of sequencing.

In the second phase of the method, samples that are used for determining the tumor fraction of the patient include samples that contain nucleic acids that are cell-free. Cell-free nucleic acids, including cfDNA, can be obtained by various methods from biological samples including but not limited to plasma, serum, and urine. Other biological fluid samples include, but are not limited to blood, sweat, tears, sputum, ear flow, lymph, saliva, cerebrospinal fluid, ravages, bone marrow suspension, vaginal flow, transcervical lavage, brain fluid, ascites, milk, secretions of the respiratory, intestinal and genitourinary tracts, amniotic fluid, milk, and leukophoresis samples. In some embodiments, the sample is a sample that is easily obtainable by non-invasive procedures, e.g., blood, plasma, serum, sweat, tears, sputum, urine, ear flow, saliva or feces. In certain embodiments the sample is a peripheral blood sample, or the plasma and/or serum fractions of a peripheral blood sample. In other embodiments, the biological sample is a swab or smear, a biopsy specimen, or a cell culture. In another embodiment, the sample is a mixture of two or more biological samples, e.g., a biological sample can comprise two or more of a biological fluid sample, a tissue sample, and a cell culture sample.

In various embodiments the cfDNA present in the sample can be enriched specifically or non-specifically prior to use (e.g., prior to capture and sequencing). Non-specific enrichment of sample DNA refers to the whole genome amplification of the DNA fragments of the sample that can be used to increase the level of the sample DNA prior to capture and sequencing. Non-specific enrichment can be the selective enrichment of exomes. Methods for whole genome amplification are known in the art. Degenerate oligonucleotide-primed PCR (DOP), primer extension PCR technique (PEP) and multiple displacement amplification (MDA) are examples of whole genome amplification methods. In some embodiments, the sample is unenriched for cfDNA.

As is described elsewhere herein, cfDNA is present as fragments averaging about 170 bp. Accordingly, further fragmentation of cfDNA is not needed. In some embodiments, sufficient cfDNA is obtained from a 10 ml blood sample to confidently determine the presence or absence of cancer in a patient. The blood samples used in the method provided can be of about 5 ml, about 10 ml, about 15 ml, about 20 ml, about 25 ml or more than 25 ml. Typically, 20 ml of blood plasma contains between 5,000 and 10,000 genome equivalents, and provides more than sufficient cfDNA for determining tumor fraction according to the method provided. In some embodiments, sufficient cfDNA is obtained from 10 ml to 20 ml of blood to determine tumor fraction.

To separate cfDNA from cells in a sample, various methods including, but not limited to fractionation, centrifugation (e.g., density gradient centrifugation), DNA-specific precipitation, or high-throughput cell sorting and/or other separation methods can be used. Commercially available kits for manual and automated separation of cfDNA are available (Roche Diagnostics, Indianapolis, Ind., Qiagen, Germantown, MD).

cfDNA can be end-repaired, and optionally dA tailed, and double-stranded adaptors comprising sequences complementary to amplification and sequencing primers are ligated to the ends of the cfDNA molecules to enable NGS sequencing, e.g., using an Illumina platform. Additionally, each of the double-stranded adaptors further comprises a non-random barcode sequence, which serves to differentiate individual cfDNA molecules. In some embodiments, the barcode sequences are random sequences. In other embodiments, the barcode sequences are non-random barcode sequences. Non-random barcode sequences provide a significant advantage over random barcode sequences because non-random barcode sequences enable unambiguous identification of the sequencing reads described below. The nonrandom barcode sequences are designed specifically to be base-balance both within and across all barcodes. Additionally, in some embodiments, the nonrandom barcodes can comprise a T nucleotide at 3′ end, which is complementary to the A nucleotide of dA-tailed cfDNA molecules. In embodiments utilizing a T nucleotide overhang at 3′ end of the barcode, barcodes of three different lengths can be designed to avoid a single base flashing across the entire flowcell of the sequencer. Nonrandom barcode sequences can be present in adaptors as sequences of 13, 14, and 15 bp; 10, 11, and 12 bp; 11, 12, and 13 bp; 13, 14, and 15 bp; 14, 15, and 16 bp; 15, 16, and 17 bp, and the like. In some embodiments, the shortest barcode sequence can be 8 bp and the longest barcode sequence can be 100 bp.

Each sequence of the somatic variants or subset panel that is present in the cfDNA sample is targeted by one or more capture probes described elsewhere herein, and is isolated for further analysis.

b. Sequencing and Analysis

The disclosed methods generally comprise sequencing one or more samples. Sequencing methods include, but are not limited to, Maxam-Gilbert sequencing-based techniques, chain-termination-based techniques, shotgun sequencing, bridge PCR sequencing, single-molecule real-time sequencing, ion semiconductor sequencing (Ion Torrent sequencing), nanopore sequencing, pyrosequencing (454), sequencing by synthesis, sequencing by ligation (SOLID sequencing), sequencing by electron microscopy, dideoxy sequencing reactions (Sanger method), massively parallel sequencing, polony sequencing, duplex sequencing, and DNA nanoball sequencing. In some embodiments, sequencing involves hybridizing a primer to the template to form a template/primer duplex, contacting the duplex with a polymerase enzyme in the presence of a detectably labeled nucleotides under conditions that permit the polymerase to add nucleotides to the primer in a template-dependent manner, detecting a signal from the incorporated labeled nucleotide, and sequentially repeating the contacting and detecting steps at least once, wherein sequential detection of incorporated labeled nucleotide determines the sequence of the nucleic acid. In some embodiments, the sequencing comprises obtaining paired end reads. The accuracy or average accuracy of the sequence information may be greater than 80%, 90%, 95%, 99% or 99.98%. In some embodiments, the sequence information obtained is more than 50 bp, 100 bp or 200 bp. The sequence information may be obtained in less than 1 month, 2 weeks, 1 week 1 day, 3 hours, 1 hour, 30 minutes, 10 minutes, or 5 minutes. The sequence accuracy or average accuracy may be greater than 95% or 99%. Examples of detectable labels include radiolabels, florescent labels, enzymatic labels, etc. In some embodiments, the detectable label may be an optically detectable label, such as a fluorescent label. Examples of fluorescent labels include cyanine, rhodamine, fluorescien, coumarin, BODIPY, alexa, or conjugated multi-dyes. In some embodiments, the nucleotide is flagged if one or more of its sequence segments are substantially similar to one or more sequence segments of another nucleotide within the same partition.

Some methods of sequencing may require or involve a prior target enrichment step. For example, use of on-sequencer enrichment, such as with a nanopore sequencer, allows for the simultaneous enrichment and sequencing of the sequence library by real-time rejection of molecules that are not from the region of interest. Alternatively, sequences can be selectively and preferentially sequenced from the region of interest.

Captured sequences can be analyzed using the sequencing-by-synthesis technology of Illumina, which uses fluorescent reversible terminator deoxyribonucleotides. The reads generated by the sequencing process are aligned to a reference sequence and associated with a sequence of the somatic sequence panel specific for the patient. Mapping of the sequence reads can be achieved by comparing the sequence of the reads with the sequence of the reference genome to determine the specific genetic information, and optionally the chromosomal origin of the sequenced nucleic acid (e.g., cfDNA) molecule. A number of computer algorithms are available for aligning sequences, including without limitation BLAST (Altschul et al., 1990), BLITZ (MPsrch) (Sturrock & Collins, 1993), FASTA (Person & Lipman, 1988), BOWTIE (Langmead et al, Genome Biology 10:R25.1-R25.10 [2009]), or ELAND (Illumina, Inc., San Diego, Calif., USA). In one embodiment, the sequencing data is processed by bioinformatic alignment analysis for the Illumina Genome Analyzer, which uses the Efficient Large-Scale Alignment of Nucleotide Databases (ELAND) software. Additional software includes SAMtools (SAMtools, Bioinformatics, 2009, 25(16):2078-9), and the Burroughs-Wheeler block sorting compression procedure which involves block sorting or preprocessing to make compression more efficient.

The barcoded cfDNA fragments isolated form the patient's fluid sample, e.g., blood sample, can be amplified, e.g., by PCR, and captured using the hybrid probes. Capturing of the barcoded fragments comprises obtaining single strands of barcoded cfDNA, and hybridizing the barcoded cfDNA with different hybrid probes. Each of the different hybrid probes hybridizes to a single-stranded barcoded cfDNA target sequence to form a target-hybrid probe duplex. The duplex is isolated from unhybridized cfDNA by binding the purification binding moiety comprised in the hybrid probe to the corresponding purification moiety binding partner. As described elsewhere herein, the corresponding purification moiety binding partner can be immobilized on a solid surface, e.g., a magnetic bead, which facilitates the separation of the capture duplex from unhybridized cfDNA molecules in solution. The barcoded cfDNA of the duplex is released, and is subjected to sequencing using an NGS instrument.

The error rate in sequencing using NGS methods is of approximately 1 in 500 bases which results in many sequencing errors. The high error rate becomes problematic especially when attempting to identify somatic mutations in mixtures of DNA sequences comprising only a small fraction of mutated species or sequences comprising single nucleotide variants. The methods described herein avoid such errors by analyzing target sequences that comprise somatic mutations having multiple changes relative to a reference sequence. Additionally, NGS methods typically utilize single stranded DNA as the primary source of sequencing material. Any error included during the amplification step of the DNA molecule prior to sequencing is perpetuated, and becomes indistinguishable as an extraneous technology-dependent mistake. Chemical errors occur at a frequency of approximately in 1000 bases. The combination of sequencing and chemical errors obscure the limit of detection (LOD).

Accordingly, in some embodiments, double-stranded sequencing of the cfDNA is performed. As described elsewhere herein cfDNA can be end-repaired, and optionally dA tailed, and double-stranded adaptors comprising sequences complementary to amplification and sequencing primers are ligated to the ends of the cfDNA molecules to enable NGS sequencing, e.g., using an Illumina platform.

The tumor fraction can then be calculated as the proportion of different cfDNA sequences each comprising at least one somatic mutation, i.e., ctDNA sequences, relative to the total number of different cfDNA, i.e., ctDNA and corresponding normal sequences. Unlike the single-stranded approach, the current method corrects for random sequencing errors.

c. Molecular Barcodes

In some embodiments, an identifier sequence, i.e., a molecular barcode, may be used to identify unique DNA molecules or target sequences in a DNA library. Molecular barcodes aid in reconstruction of a contiguous DNA sequences or assist in copy number variation determination. Exemplary markers include nucleic acid binding proteins, optical labels, nucleotide analogs, nucleic acid sequences, and others known in the art.

In some embodiments, the molecular barcode is a nanostructure barcode. In some embodiments, the molecular barcode comprises a nucleic acid sequence that when joined to a target polynucleotide serves as an identifier of the sample or sequence from which the target polynucleotide was derived. In some embodiments, molecular barcodes are at least 3, 4, 5, 6, 7, 8, 9, 10, 11, 12, 13, 14, 15, or more nucleotides in length. In some embodiments, molecular barcodes are shorter than 10, 9, 8, 7, 6, 5, or 4 nucleotides in length. In some embodiments, each molecular barcode in a plurality of molecular barcodes differ from every other molecular barcode in the plurality at least three nucleotide positions, such as at least 3, 4, 5, 6, 7, 8, 9, 10, or more positions. In some embodiments, molecular barcodes associated with some polynucleotides are of different length than molecular barcodes associated with other polynucleotides. In general, molecular barcodes are of sufficient length and comprise sequences that are sufficiently different to allow the identification of samples based on molecular barcodes with which they are associated. In some embodiments, both the forward and reverse adapter comprise at least one of a plurality of molecular barcode sequences. In some embodiments, each reverse adapter comprises at least one of a plurality of molecular barcode sequences, wherein each molecular barcode sequence of the plurality of molecular barcode sequences differs from every other molecular barcode sequence in the plurality of molecular barcode sequences.

In some embodiments, every molecular barcode in a set is unique, that is, any two molecular barcodes chosen out of a given set will differ in at least one nucleotide position. Furthermore, it is contemplated that molecular barcodes have certain biochemical properties that are selected based on how the set will be used. For example, certain sets of molecular barcodes that are used in an RT-PCR reaction should not have complementary sequences to any sequence in the genome of a certain organism or set of organisms. A requirement for non-complementarity helps to ensure that the use of a particular molecular barcode sequence will not result in mis-priming during molecular biological manipulations requiring primers, such as reverse transcription or PCR. Certain sets satisfy other biochemical properties imposed by the requirements associated with the processing of the sequence molecules into which the barcodes are incorporated.

Examples of sequencing technologies for sequencing molecular barcodes, as well as any generated nucleotide-based sequence, include, but are not limited to, Maxam-Gilbert sequencing-based techniques, chain-termination-based techniques, shotgun sequencing, bridge PCR sequencing, single-molecule real-time sequencing, ion semiconductor sequencing (Ion Torrent sequencing), nanopore sequencing, pyrosequencing (454), sequencing by synthesis, sequencing by ligation (SOUD sequencing), sequencing by electron microscopy, dideoxy sequencing reactions (Sanger method), massively parallel sequencing, polony sequencing, and DNA nanoball sequencing.

In some embodiments, molecular barcodes are used to improve the power of copy-number calling algorithms by reducing non-independence from PCR duplication. In another embodiment, molecular barcodes can be used to improve test specificity by reducing sequence error generated during amplification.

### V. CORRECTION BASED ON TUMOR ALLELE BALANCE, DROP RATE, LATENT VARIANT CLASSES AND SAMPLE OR PANEL-SPECIFIC ERROR RATES

Although MRD methods generally provide significant clinical utility in tracking treatment, recurrence, and prognosis of cancer patients, certain aspects of prior MRD processes can be improved with the disclosed methods. Specifically, inference of ctDNA or MRD status can be challenging in low molecular depth sequencing or because of sample and/or somatic variant background error. The present disclosure is the first to recognize that accounting for, low molecular depth sequencing and sample and somatic variant background error and subsequently correcting therefrom improves the detection of MRD.

The goal of a minimum residual disease (MRD) assay is to detect and quantify circulating tumor DNA so researchers and clinicians can detect recurrence early and monitor the progress of the disease through treatment. For a tumor-informed panel the general steps are to (1) profile the tumor, (2) identify a subset of somatic sites to target, (3) enrich cell-free DNA (cfDNA) for target sites, and (4) estimate the tumor content of the cfDNA given the tumor profile and sequencing data. The methods described herein can include computational methods for the last step-tumor fraction quantification.

As described in more detail in the Examples, the disclosed methods may utilize various types of modeling in the analysis of cfDNA analysis in which a candidate set of somatic variants identified in tumor-normal sequencing is modeled as a mixture of variants coming from the tumor, non-tumor somatic variants (e.g., CHIP mutations), error-prone sites, and germline variants. For each variant “class” a likelihood function was developed describing the probability of observing the ALT-bearing reads given the total set of reads at a site if the variant is in a given class. The total likelihood of the data for each variant is the average of the per-class likelihoods weighted by the proportion of variants in each class, and the total likelihood of the data for all variants is the product of per-variant likelihoods. Only the tumor class includes tumor fraction as a parameter of the likelihood function, so fitting the model allows quantification of tumor fraction while marginalizing out the signal from non-tumor variants, which reduces the chance of a false positive when the target set includes non-tumor variants.

For the purposes of the methods described herein, all class likelihoods were modeled as binomial on the count of variant-bearing molecules given the total molecular depth at a site. The “probability” parameter of the binomial is set to 0.5 for the germline class, 0.01-0.1 for the CHIP+error-prone class (i.e., the “noise” class), and for the tumor class the probability parameter is a freely varying value in (0,1) calculated as a function of the copy number and genotype in the tumor at each target site. Future iterations could expand on the per-class likelihood functions by introducing information about insert sizes or molecule start sites, both of which can help differentiate tumor from germline or noise sites. As explained further below, the model is fit with expectation-maximization.

Accordingly, the disclosed methods related to improving the detection, monitoring, and treatment of a cancer patient undergoing MRD assessment. The patient can be suspected or known to harbor a solid tumor, or the patient may have previously harbored a solid tumor. In some aspects the solid tumor is a tumor of a tissue or organ. In other aspects, the solid tumor is a metastatic mass of a blood borne cancer. The present methods can also be applicable to the detection and/or monitoring of blood borne or hematological cancers.

The disclosed methods are applicable to MRD testing, wherein the patient has previously been treated for a cancer, and may be considered in remission, however a small number of cancer cells remain in the body. The number of remaining cells may be so small that they do not cause any physical signs or symptoms and often cannot even be detected through traditional methods, such as viewing cells under a microscope and/or by tracking abnormal serum proteins in the blood. An MRD positive test results means that residual (remaining) disease was detected. A negative result means that residual disease was not detected. MRD testing may be used to measure the effectiveness of treatment and to predict if a patient is at risk of relapse. When a patient tests positive for MRD, it means that there are still residual cancer cells in the body after treatment. When MRD is detected, this is known as “MRD positivity.” When a patient tests negative, no residual cancer cells were found. When no MRD is detected, this is known as “MRD negativity.”

Current MRD methods are limited by the amount of blood that can be drawn for analysis and by the extremely low proportions of tumor cfDNA of about 1e-4. The methods provided herein combine analysis of patient-specific somatic variants, e.g., single nucleotide variants (SNVs), multi-nucleotide variants (MNVs), insertions (e.g., insertion of one or more nucleotides at a locus but less than the entire locus), deletions (e.g., deletion of one or more nucleotides at a locus), inversions (e.g., reversal of a sequence of one or more nucleotides), an genomic rearrangements (e.g., deletions, duplications, inversions, and translocations), which allows the detection of somatic variants associated with the patient's cancer at extremely low proportions of tumor cfDNA of less than about 1e-3.

The disclosed methods utilizes correction based on tumor allele balance using tumor purity, sites that do not contribute to ctDNA signal in plasma (e.g., dropout rate), and/or inclusion of latent variant classes to account for somatic calling errors. Additionally, correction based on defining and accounting for sample and/or panel-specific error rates.

a. Correction Based on Allele Balance

Correction of detecting the presence or absence of ctDNA sequences in the sequencing library can be performed by correcting for tumor allele balance, which is a ratio of variant allele to reference allele in sequencing data, using tumor purity. Tumor samples can be contaminated with normal tissue, either due to errors in dissection or immune cell infiltration. This can reduce the observed allele balance for somatic variant sites, resulting in an artificially inflated inferred tumor fraction overall. This approach is modeled on the relationship between the probability of observing alternative allele counts, p, at a site given allele balance in the tumor (AB_{T}) copy number of the site in both tumor (CN_{T}) and normal (CN_{N}) and the fraction of tumor DNA in the sample, the tumor purity (TP) and the error rate estimated from control site data (err):

$$p = \frac{T F \cdot {CN}_{T}}{T F \cdot {CN}_{T} + ( 1 - T F ) \cdot {CN}_{N}} \cdot {AB}_{T} / T P + err$$

Each variant will be ranked by an expected p of 0.005% TF using allele balance and copy number in the tumor sample, assuming copy number in the non-tumor sample is 2. This approach accounts for the sub-clonal variants on detection sensitivity.
b. Correction for Dropout Rate

Correction of detecting the presence or absence of ctDNA sequences in the sequencing library can be performed by correcting for sites that do not contribute to ctDNA signal. Such sites include sites due to biological reasons (e.g., lack of shedding into the blood stream) or technical reasons (e.g., false positive somatic variant call). These factors are collectively referred to as a dropout rate (DR) which is the false discovery rate (FDR)+ (1−FDR) multiplied by biological false negative rate (alternatively plasma dropout rate) (PDR):

$$D R = F D R + ( 1 - F D R ) \cdot P D R$$

The dropout rate (DR) is used to weight a binomial mixture model of the base model+an error-only model:

$$arg max [ {\prod }_{i = 1}^{j} ( 1 - D R ) L ( {p}_{i} \vert {n}_{i} , {k}_{i} ) + D R \cdot L ( err \vert {n}_{i} , {k}_{i} ) ] ,$$

$$where : L ( p \vert n , k ) = P r ( n , k \vert p ) and k = ALT counts$$

Whereby a higher likelihood ration indicates a greater confidence in tumor recurrence:

$$L R = \frac{L ( p \vert n , k )}{L ( e r r \vert n , k )}$$

c. Correction for Latent Variant Classes

Correction of detecting the presence or absence of ctDNA sequences in the sequencing library can be performed by correcting for sites that do not contribute to ctDNA signal. Occasionally, germline variants, sites with systematic noise, low frequency clonal hematopoiesis of indeterminate potential (CHIP) variants or other anomalous sites may be selected. Detection of such sites can negatively influence the fit of the base model and result in a false positive MRD status. To avoid an incorrect fit of the base+dropout mixture model, the mixture model can be expanded to include correction for latent variant classes.

d. Correction for Sample or Panel-Specific Error Rates

A common approach to error-reduction in next generation sequencing (NGS) is the use of unique molecular identifiers (UMIs), tagging individual DNA molecules with a unique barcode that are associated with all sequences deriving from that molecule. Sequences with the same barcode are collapsed to form a consensus sequence that minimizes random sequencing and late cycle PCR errors. However, early cycle PCR errors are less likely to be corrected by this UMI approach. The presence of this residual error interferes with the interpretation of somatic variant detection from samples with minute fractions of ctDNA. For each site included in the sequence library, a control site or a set of control sites can be selected. Control site can be anywhere within the sequence library or in any sequence reads, or are local to the somatic variant site, within about, for example, 160 nucleotides, 120 nucleotides, or 20 nucleotides and have the same reference base as the somatic variant site. In some embodiments, the control site is located within about 1 nucleotide to about 3 nucleotides upstream or downstream. At the control sites, counts of the reference and variant bases are collected. For each mutation type, (e.g., A>G) a statistical model (e.g., a binomial model) for error rate can fit by maximum likelihood estimation. The mutation type-specific error rates are weighted by the proportion of that mutation type included in the MRD panel to derive a sample/panel-specific error rate. That rate can be used as the background probability of detecting a variant allele in a tumor fraction inference model. The advantage of defining a sample/panel-specific error rate is that it controls for differences in how samples were collected and processed (e.g., if PCR conditions vary slightly between NGS library preparations) as well as differences in composition of panels between patients (e.g., if a panel include more of an error-prone mutation type).

The disclosed methods are also applicable to MRD testing, wherein the patient has previously been treated for a cancer, and may be considered in remission, however a small number of cancer cells remain in the body. The number of remaining cells may be so small that they do not cause any physical signs or symptoms and often cannot even be detected through traditional methods, such as viewing cells under a microscope and/or by tracking abnormal serum proteins in the blood. An MRD positive test results means that residual (remaining) disease was detected. A negative result means that residual disease was not detected. MRD testing may be used to measure the effectiveness of treatment and to predict if a patient is at risk of relapse. When a patient tests positive for MRD, it means that there are still residual cancer cells in the body after treatment. When MRD is detected, this is known as “MRD positivity.” When a patient tests negative, no residual cancer cells were found. When no MRD is detected, this is known as “MRD negativity.”

For the purposes of the disclosed methods, the variants that are targeted for enrichment are generally somatic variants; however, the variants may also include de novo genetic variant. That is, if the genetic variant is not present in non-cancerous cells of the cancer patient, and the described method indicates that the genetic variant is distinguishable from the cancer patient genome, then the genetic variant is a de novo variant. Accordingly, some embodiments of the disclosed methods may comprise determining whether a genetic variant is an inherited genetic variant or a de novo genetic variant.

In a second phase, monitoring of the status of the cancer in the patient is performed using the patient's panel of capture probes to identify somatic mutations that are circulating as cfDNA. The second phase is non-invasive and requires clinically viable amounts of a biological fluid, e.g., a peripheral blood draw of about 5-25 ml (e.g., about 5, about 10, about 15, about 20, or about 25 mls), which can be repeated as frequently as desired to detect changes in the patient's cancer. A clinically viable amount of biological fluid, e.g., whole blood, typically comprises at least 1000 genome equivalents, at least 2000 genome equivalents, at least 3000 genome equivalents, at least 4000 genome equivalents, at least 5000 genome equivalents, at least 6000 genome equivalents, at least 7000 genome equivalents, at least 8000 genome equivalents, at least 9000 genome equivalents, at least 10000 genome equivalents, at least 11000 genome equivalents, at least 12000 genome equivalents, or at least 15000 genome equivalents. In some embodiments, the second phase of the method utilizes a whole blood sample of between 5 ml and 20 ml, comprising between 3000 and 15000 genome equivalents.

First, a panel of sequences comprising somatic mutations specific to the tumor of a patient is identified as follows. DNA (e.g., genomic DNA or cfDNA) is isolated from the tumor and from a non-tumor sample, such as normal tissue (i.e., non-cancerous tissue) or whole blood, and sequenced. DNA sequences from the tumor and non-tumor samples are compared, and a set of somatic mutations specific to the patient's tumor are identified.

The subset of somatic variants serves as a signature panel for the patient that can be sequenced at various stages of the disease, i.e., the signature panel can be screened to determine the presence of cancer at surgery following diagnosis; during cancer treatment, e.g., at intervals during chemotherapy or radiation therapy, to monitor the efficacy of the treatment; at intervals during remission to confirm continued absence of disease; and/or to detect recurrence of the disease. The composition of the selected somatic variants for the subset is a key determinant for the sensitivity and specificity of the methods described herein.

Next, a set of capture probes is obtained (e.g., probes). The set of capture probes comprises sequences that are capable of hybridizing to specific target sequences in the patient's genome and that encompass the sites comprising the somatic variants identified in the tumor tissue. In some embodiments, the capture probe comprises sequences that are capable of hybridizing to specific target sequences and a control site. In some embodiments, the control site is located anywhere within the sequence library or in any sequence reads. In some embodiments, the corresponding control site for each somatic variant is located within 20 nucleotides of the somatic variant. In some embodiments, the control site is located about 1 nucleotide to about 3 nucleotides of the somatic variant. However, it will be appreciated that the control site does not have to be proximate to the corresponding target sequence or somatic mutations. Rather, the control site generally comprises a reference base that is the same as the base of the somatic variant. For example, control sites can be selected that match a given reference base (e.g., A), and the error rate of a particular variant type (e.g., A>G) can be determined across any of the sequence reads. By checking for changes at the control site(s), regardless of proximity to the target sequence or somatic mutations, one can obtain a maximum likelihood estimation for an error (e.g., a sequencing error or amplification/PCR error) that results in a given type of change in a sample. This could be done, for example, using a statistical model, such as a binomial model. This error rate can then be used as background for all target variants or variant alleles of the same type.

Subsequently, the presence of ctDNA and/or the tumor fraction in a fluid sample from the same patient is determined. Determining the tumor fraction comprises obtaining cfDNA from the patient, and using the capture probes designed for the patient-specific subset panel to capture cfDNA target sequences comprising tumor sequences (i.e., ctDNA). The captured DNA is sequenced, and the sequences can be analyzed and enumerated. The tumor fraction can be determined by fitting a binomial mixture model of variant counts and total counts across the entire panel of variants assayed, where the mixture components are the tumor, non-tumor, or germline variants and the weighting of each class is determined by the probability that a variant belongs to that class. Enumeration of mutated and unmutated allelic sequences can be accomplished by analyzing the countable sequence reads obtained from the sequencing process. The method does not necessitate that all somatic mutations in the patient's signature panel be detected. Rather, a test or assay can be considered positive (i.e., ctDNA is present) if as little as a single somatic mutation in the patient's signature panel is detected.

Alternatively, the sequences can be analyzed and a control site can be selected in the sequence library for each somatic variant where the control site matches a reference base for the corresponding somatic variant. An error rate for a variant allele of the reference base can be calculated by detecting changes at the control site, where the error rate is a background probability of detecting the variant allele and the presence or absence of ctDNA sequences in the sequence library is detected. The control site can be located anywhere within the sequence library or in any sequence reads. The control site can be located within about 160 bases, about 150 bases, about 140 bases, about 130 bases, about 120 bases, about 110 bases, about 100 bases, about 90 bases, about 80 bases, about 70 bases, about 60 bases, about 50 bases, about 40 bases, about 30 bases, about 20 bases, about 10 bases, about 5 bases, about 3 bases, or about 1 base.

The tumor fraction can be determined as the proportion of sequences comprising a somatic mutation of the total number of mutated and corresponding unmutated allelic sequences. Enumeration of mutated and unmutated allelic sequences can be accomplished by analyzing the countable sequence reads obtained from the sequencing process. The method does not necessitate that all somatic mutations in the patient's signature panel be detected. Rather, a test or assay can be considered positive (i.e., ctDNA is present) if as little as a single somatic mutation in the patient's signature panel is detected.

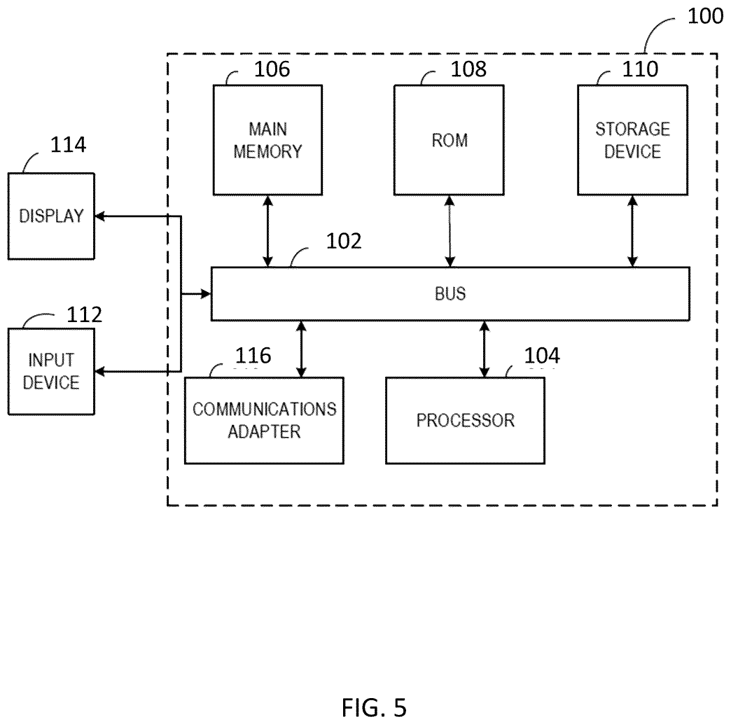

FIG. 5 is a component diagram of an example computing system suitable for use in the various implementations described herein, according to an example implementation. One or more steps of the methods and processes discussed herein can be performed by the computing system depicted in FIG. 5.

The computing system 100 includes a bus 102 or other communication component for communicating information and a processor 104 coupled to the bus 102 for processing information. The computing system 100 also includes main memory 106, such as a RAM or other dynamic storage device, coupled to the bus 102 for storing information, and instructions to be executed by the processor 104. Main memory 106 can also be used for storing position information, temporary variables, or other intermediate information during execution of instructions by the processor 104. The computing system 100 may further include a ROM 108 or other static storage device coupled to the bus 102 for storing static information and instructions for the processor 104. A storage device 110, such as a solid-state device, magnetic disk, or optical disk, is coupled to the bus 102 for persistently storing information and instructions.

The computing system 100 may be coupled via the bus 102 to a display 114, such as a liquid crystal display, or active matrix display, for displaying information to a user. An input device 112, such as a keyboard including alphanumeric and other keys, may be coupled to the bus 102 for communicating information, and command selections to the processor 104. In another implementation, the input device 112 has a touch screen display. The input device 112 can include any type of biometric sensor, or a cursor control, such as a mouse, a trackball, or cursor direction keys, for communicating direction information and command selections to the processor 104 and for controlling cursor movement on the display 114.

In some implementations, the computing system 100 may include a communications adapter 116, such as a networking adapter. Communications adapter 116 may be coupled to bus 102 and may be configured to enable communications with a computing or communications network or other computing systems. In various illustrative implementations, any type of networking configuration may be achieved using communications adapter 116, such as wired (e.g., via Ethernet), wireless (e.g., via Wi-Fi, Bluetooth), satellite (e.g., via GPS) pre-configured, ad-hoc, LAN, WAN, and the like.

According to various implementations, the processes of the illustrative implementations that are described herein can be achieved by the computing system 100 in response to the processor 104 executing an implementation of instructions contained in main memory 106. Such instructions can be read into main memory 106 from another computer-readable medium, such as the storage device 110. Execution of the implementation of instructions contained in main memory 106 causes the computing system 100 to perform the illustrative processes described herein. One or more processors in a multi-processing implementation may also be employed to execute the instructions contained in main memory 106. In alternative implementations, hard-wired circuitry may be used in place of or in combination with software instructions to implement illustrative implementations. Thus, implementations are not limited to any specific combination of hardware circuitry and software.

The implementations described herein have been described with reference to drawings. The drawings illustrate certain details of specific implementations that implement the systems, methods, and programs described herein. However, describing the implementations with drawings should not be construed as imposing on the disclosure any limitations that may be present in the drawings.

It should be understood that no claim element herein is to be construed under the provisions of 35 U.S.C. § 112 (f), unless the element is expressly recited using the phrase “means for.”

As used herein, the term “circuit” may include hardware structured to execute the functions described herein. In some implementations, each respective “circuit” may include machine-readable media for configuring the hardware to execute the functions described herein. The circuit may be embodied as one or more circuitry components including, but not limited to, processing circuitry, network interfaces, peripheral devices, input devices, output devices, sensors, etc. In some implementations, a circuit may take the form of one or more analog circuits, electronic circuits (e.g., integrated circuits (IC), discrete circuits, system on a chip (SOC) circuits), telecommunication circuits, hybrid circuits, and any other type of “circuit.” In this regard, the “circuit” may include any type of component for accomplishing or facilitating achievement of the operations described herein. For example, a circuit as described herein may include one or more transistors, logic gates (e.g., NAND, AND, NOR, OR, XOR, NOT, XNOR), resistors, multiplexers, registers, capacitors, inductors, diodes, wiring, and so on.

The “circuit” may also include one or more processors communicatively coupled to one or more memory or memory devices. In this regard, the one or more processors may execute instructions stored in the memory or may execute instructions otherwise accessible to the one or more processors. In some implementations, the one or more processors may be embodied in various ways. The one or more processors may be constructed in a manner sufficient to perform at least the operations described herein. In some implementations, the one or more processors may be shared by multiple circuits (e.g., circuit A and circuit B may comprise or otherwise share the same processor, which, in some example implementations, may execute instructions stored, or otherwise accessed, via different areas of memory). Alternatively or additionally, the one or more processors may be structured to perform or otherwise execute certain operations independent of one or more co-processors.

In other example implementations, two or more processors may be coupled via a bus to enable independent, parallel, pipelined, or multi-threaded instruction execution. Each processor may be implemented as one or more general-purpose processors, ASICs, FPGAs, GPUs, TPUs, digital signal processors (DSPs), or other suitable electronic data processing components structured to execute instructions provided by memory. The one or more processors may take the form of a single core processor, multi-core processor (e.g., a dual core processor, triple core processor, or quad core processor), microprocessor, etc. In some implementations, the one or more processors may be external to the apparatus, for example, the one or more processors may be a remote processor (e.g., a cloud-based processor). Alternatively or additionally, the one or more processors may be internal or local to the apparatus. In this regard, a given circuit or components thereof may be disposed locally (e.g., as part of a local server, a local computing system) or remotely (e.g., as part of a remote server such as a cloud based server). To that end, a “circuit” as described herein may include components that are distributed across one or more locations.

An exemplary system for implementing the overall system or portions of the implementations might include a general purpose computing devices in the form of computers, including a processing unit, a system memory, and a system bus that couples various system components including the system memory to the processing unit. Each memory device may include non-transient volatile storage media, non-volatile storage media, non-transitory storage media (e.g., one or more volatile or non-volatile memories), etc. In some implementations, the non-volatile media may take the form of ROM, flash memory (e.g., flash memory such as NAND, 3D NAND, NOR, 3D NOR), EEPROM, MRAM, magnetic storage, hard discs, optical discs, etc. In other implementations, the volatile storage media may take the form of RAM, TRAM, ZRAM, etc. Combinations of the above are also included within the scope of machine-readable media. In this regard, machine-executable instructions comprise, for example, instructions and data, which cause a general-purpose computer, special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, script components), in accordance with the example implementations described herein.

It should also be noted that the term “input devices,” as described herein, may include any type of input device including, but not limited to, a keyboard, a keypad, a mouse, joystick, or other input devices performing a similar function. Comparatively, the term “output device,” as described herein, may include any type of output device including, but not limited to, a computer monitor, printer, facsimile machine, or other output devices performing a similar function.

It should be noted that although the diagrams herein may show a specific order and composition of method steps, it is understood that the order of these steps may differ from what is depicted. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative implementations. Accordingly, all such modifications are intended to be included within the scope of the present disclosure as defined in the appended claims. Such variations will depend on the machine-readable media and hardware systems chosen and on designer choice. It is understood that all such variations are within the scope of the disclosure. Likewise, software and web implementations of the present disclosure could be accomplished with standard programming techniques with rule-based logic and other logic to accomplish the various database searching steps, correlation steps, comparison steps, and decision steps.

While this specification contains many specific implementation details, these should not be construed as limitations on the scope of any inventions or of what may be claimed, but rather as descriptions of features specific to particular implementations of the systems and methods described herein. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Having now described some illustrative implementations and implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements, and features discussed only in connection with one implementation are not intended to be excluded from a similar role in other implementations.

The phraseology and terminology used herein is for the purpose of description and should not be regarded as limiting. The use of “including,” “comprising,” “having,” “containing,” “involving,” “characterized by,” “characterized in that,” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act, or element may include implementations where the act or element is based at least in part on any information, act, or element.

Any implementation disclosed herein may be combined with any other implementation, and references to “an implementation,” “some implementations,” “an alternate implementation,” “various implementation,” “one implementation,” or the like are not necessarily mutually exclusive and are intended to indicate that a particular feature, structure, or characteristic described in connection with the implementation may be included in at least one implementation. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms.

Where technical features in the drawings, detailed description or any claim are followed by reference signs, the reference signs have been included for the sole purpose of increasing the intelligibility of the drawings, detailed description, and claims. Accordingly, neither the reference signs nor their absence have any limiting effect on the scope of any claim elements.

The foregoing description of implementations has been presented for purposes of illustration and description. It is not intended to be exhaustive or to limit the disclosure to the precise form disclosed, and modifications and variations are possible in light of the above teachings or may be acquired from this disclosure. The implementations were chosen and described in order to explain the principals of the disclosure and its practical application to enable one skilled in the art to utilize the various implementations and with various modifications as are suited to the particular use contemplated. Other substitutions, modifications, changes, and omissions may be made in the design, operating conditions and implementation of the implementations without departing from the scope of the present disclosure as expressed in the appended claims.

### VI. EXAMPLES

The present invention is described in further detail in the following examples which are not in any way intended to limit the scope of the invention as claimed. All references cited are herein specifically incorporated by reference for all that is described therein. The following examples are offered to illustrate, but not to limit the claimed invention.

### Example 1: Improved Sensitivity and Estimation of MRD-Correcting for Allele Balance of the Somatic Variants Based on Purity of the Tumor Sample, a Dropout Rate of Sites, and/or Inclusion of Latent Variant Classes

Preparing somatic variant panel: DNA is extracted from a normal and tumor sample from a patient and a sequencing library is prepared for each sample. The samples are sequenced by whole genome sequencing and somatic variants identified. A panel of the somatic variants is then selected by selecting sites having an expected variant allele frequency based on copy number and allele balance reference cut off and/or a plurality of features selected by a machine learning model trained with whole genome sequencing data. Hybrid capture probes are then generated for the somatic variants of the subpanel.

Correction of ctDNA sequences: cfDNA is extracted from the patient and selectively enriched using hybrid capture probes for the somatic variants panel. The enriched library is sequenced to generate sequencing reads for each of the somatic variant panel. A computer processor is used to calculate the allele balance of the somatic variants based on purity of the tumor sample, a dropout rate of sites that do not contribute to a ctDNA signal, and/or inclusion of latent variant classes in the sequence library by comparing the sequence library to a reference panel such as a known buddy coat sequence).

Diagnosing MRD: MRD is diagnosed based on the presence of the corrected patient specific somatic variants in the cfDNA sample.

### Example 2: Improved Sensitivity and Estimation of MRD-Correcting Sample or Panel Specific Error Rates

Preparing somatic variant panel: DNA is extracted from a normal and tumor sample from a patient and a sequencing library is prepared for each sample. The samples are sequenced by whole genome sequencing and somatic variants identified. A panel of the somatic variants is then selected by selecting sites having an expected variant allele frequency based on copy number and allele balance reference cut off and/or a plurality of features selected by a machine learning model trained with whole genome sequencing data. Hybrid capture probes are then generated for the somatic variants of the subpanel.

Correction using a control site: cfDNA is extracted from the patient and selectively enriched using hybrid capture probes for the somatic variant and a corresponding control site, where each corresponding control site for each somatic variant is located within 20 nucleotide bases of the somatic variant on the DNA fragment and each corresponding control site comprises a reference based that is the same as the base of the somatic variant. The enriched library is then sequenced to generate sequencing reads for each of the somatic variant panel.

Diagnosing MRD: MRD is diagnosed based on the presence of the corrected patient specific somatic variants in the cfDNA sample.

### Example 3: Computational Methods for Tumor Fraction Quantification

a. Variables

The data considered in this model includes:
Xᵢ: the count of ALT alleles at a somatic target site i; and
nᵢ: the total sequencing depth at a somatic target site i.

Additional data is passed from the “tumor profile” step:
c_{t,i}: the copy number of the tumor at site i;
a_{t,i}: the allele balance of the target allele in the tumor FFPE sample at site i (note this is what is observed in the sample before accounting for tumor purity);
tₚ: the tumor purity, or genome-equivalents proportion of DNA from the FFPE tumor sample coming from tumor vs patient normal tissue;
c_{n,i}: the copy number of the patient normal at site i. For convenience it is assumed that this is 2 on all autosomes and either 1 or 2 on allosomes depending on patient sex;
a_{n,i}: the allele balance of the target allele in the patient normal (buffy coat) sample at site i.

Additional variables include:
t_{f}: tumor fraction. The genome-equivalent proportion of cfDNA coming from the tumor.
a_{i,k}: the expected allele balance if variant i if it came from mixture class k.
   b. The Model

Target sites can come from three classes: (1) somatic variants in the tumor, (2) non-tumor somatic variants (e.g., a variant that arises from clonal hematopoiesis of indeterminate potential, a “CHIP mutation”) present in normal tissue or immune cells infiltrating the tumor FFPE sample, or (3) germline heterozygous sites incorrectly identified as somatic due to a masking failure in WGS somatic calling. The variants identified in (2) and (3) are examples of latent variants. Ideally all the variants would be from the tumor, however in practice other variants are not always dropped, and high-frequency variants significantly inflate tumor fraction estimates and detection likelihood ratios. Hard allele balance or insert size cutoffs have also been attempted to just mask non-tumor variants, but these were difficult to set in a principled way and were not successful in experiments, in which there were a number of samples with significant numbers of CHIP-like detections, accordingly, the variants should be dealt with flexibly in the model.

The likelihood of the data (X, n) as a mixture of the three classes listed above can be modeled, indexed by k. The likelihood of the data is the standard mixture model formula (equation 1):

$$L ( X \vert n , {c}_{t} , {a}_{t} , {t}_{p} ) = {\prod }_{i} {\sum }_{k} {π}_{k} L ( {X}_{i , k} \vert {n}_{i , k} , {c}_{t , i , k} , {a}_{t , i , k} , {t}_{p} )$$

πₖ variable is the “mixture weights”, or the proportion of variants coming from each k class.

L(X_{i,k}| . . . ) is the likelihood of observing X ALT reads at site i if it is in class k. The binomial probability mass function is used for all the classes, which has parameters n (the total (deduplicated) sequencing depth) and p (the probability of sampling an ALT-bearing read from the pool of molecules covering a target site).

c. Detection Probabilities (pₖ)

Each of the k mixture classes is modeled as a binomial probability B(X|n·p). The p values are the probability of success, i.e. the probability of drawing a target mutation from the pool of molecules sequenced at a site.

### Tumor DNA

If k=1 and the site is from the tumor, then the probability of observing an ALT-bearing read is the expected allele balance of the target mutation in the cell-free DNA mixture. This depends both on the proportion of tumor molecules in the cfDNA (the tumor fraction), and the proportion of target mutations in the pure tumor (itself a function of the copy number, genotype, and subclonality of the target variant, plus the tumor purity which is used to correct the observed allele balance in the FFPE sample for what it would look like if it was 100% pure tumor). The full form of this is (equation 2):

$$α = \frac{{t}_{f} {c}_{t} \frac{{a}_{t}}{{t}_{p}} + ( 1 - {t}_{f} ) {c}_{n} {a}_{n}}{{t}_{f} {c}_{t} \frac{{a}_{t}}{{t}_{p}} + ( 1 - {t}_{f} ) {c}_{n} {a}_{n} + {t}_{f} {c}_{t} ( 1 - \frac{{a}_{t}}{{t}_{p}} ) + ( 1 - {t}_{f} ) {c}_{n ( 1 -} {a}_{n} )} + e$$

where t_{f} is the tumor fraction, and e is the error rate (discussed more below). The two terms in the numerator give the number of ALT alleles expected to come from the tumor and the patient, and the four terms on the bottom give the total number of ALT or REF alleles coming from tumor or patient normal, with the goal being to get to the standard allele balance formula: α=ALT/(ALT+REF).

### CHIP Mutations

CHIP mutations are somatic variants found in white blood cells but not (necessarily) associated with the tumor. Because buffy is used as the “normal” for tumor-normal somatic calling, most high-frequency CHIP mutations should be dropped at that stage. But low-frequency CHIP mutations may get through because the typical WGS depth (˜30×) is not high enough to regularly detect somatic variants present at just 1-2%.

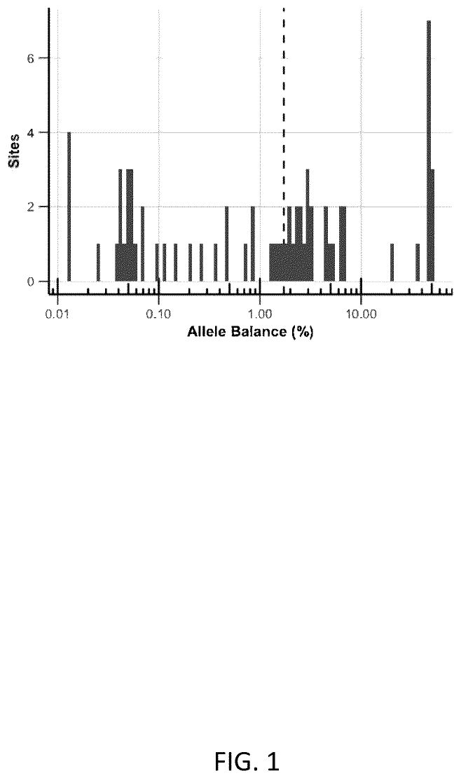

Currently, there is no principled way of modeling the expected frequency of CHIP variants. By definition, it can be said that they don't come from tumor, so t_{f}=0. But their copy number or allele balance in patient normal is not known. Empirically, looking at previously generated data, the average allele balance of target mutations detected in the buffy coat is 1.7%. Accordingly, the expected allele balance of a CHIP variant can be set at 1.7% (FIG. 1).

### Germline Variants

The somatic calling and site prioritization algorithm is designed to mask out any position variable in the patient normal, but some errors inevitably get through. This can happen, for example, in cases where there is relatively low depth on the patient normal sample which fails to sample any reads bearing one of the two alleles at a heterozygous site or homozygous site. If the variant is observed in the FFPE tumor reads, it appears as a high-frequency somatic mutation and will be upweighted by the selection algorithm. It is possible that these germline sites are interpreted as coming from the tumor, because they also have very high frequency in the cfDNA, and will therefore significantly throw off the maximum likelihood fit for tumor fraction. The germline sites can be either germline heterozygous sites, germline homozygous sites (i.e., homozygous alternative “HOMALT” targets), or a combination thereof.

A germline heterozygous site by definition has αₜ=0 and αₙ=0.5. It is also assumed that it is CN2 in tumor and normal (cₜ=2, cₙ=2). Furthermore, because it is coming from the patient normal only, it can be modeled using equation 2 with the above values filled in, giving the expected value of 0.5.

### Error Rates

On a per-read level target mutations can be observed through genuine non-biological error. The deduplication and error correction pipeline is designed to minimize these, but they still happen (currently at a rate of about 1 error per million bases). In particular, PCR errors occurring in the first few cycles are likely to get through the error correction pipeline because they will be propagated to up to half the molecules eventually sequenced and therefore a majority of error-containing molecules may end up being sampled.

The separate “error model” described here estimates this rate. Essentially, for each target mutation, all the other positions covered by the same probe matching the target reference base are found, and the number of times a read matching the target somatic mutation is observed are counted (for example, if the target is T->A at chrl: 100, all the T positions from chrl: 50-150 are found and it is determined how often a read bearing A at those positions is observed). Alternatively, all sites with the same target reference based are found, not limited to any base size range. For the purposes of the tumor fraction model an error rate value is obtained that can be plugged in to e above.

### Fitting the Model

In this example, it is assumed that e is known. The other free parameters are t_{f} (the tumor fraction), and the vector of mixture weights π.

Expectation-maximization (EM) was used to fit the model. Plausible estimations are used for the parameters of interest, e.g., t_{f}=0.001 and π=[0.95, 0.04, 0.01] (listing π in the order of [tumor, CHIP, germline]. Under these parameter values an initial likelihood matrix are filled out with one row per mixture class (tumor/CHIP/germline) and one column per site. Each entry is the binomial log-likelihood (i.e., the probability mass) of observing X ALT reads given total sequencing depth n and a probability of detection p determined by the equations above (equation 2 for tumor variants, 0.01725 for CHIPs, and 0.5 for germline variants).

The EM loop is then entered. In the “E” step the mixture weights were updated. The most likely mixture class for each variant is the class with the maximum likelihood value in each column of the likelihood matrix, which is achieved by running an argmax function down the columns. The proportion of variants in each mixture class is then calculated and used as a new set of mixture weights.

In the “M” step, the new mixture weights are used to find a new optimum tumor fraction estimate. Finally, the total likelihood of the data is calculated using equation 1 (but in log space and using the logSumExp function to approximate Σ log(B(X|n,p)).

This procedure can then be repeated an arbitrary number of times. Each iteration should improve the total likelihood, and repeats are stopped when the change in likelihood falls below a threshold.

### Example 2: Testing the EM Algorithm on a cfDNA Sample

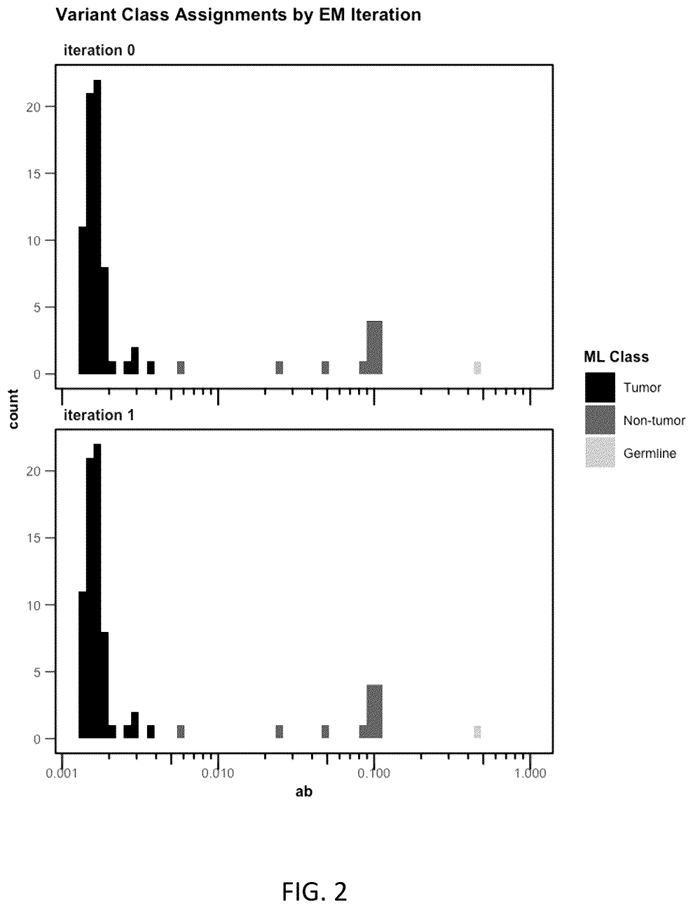

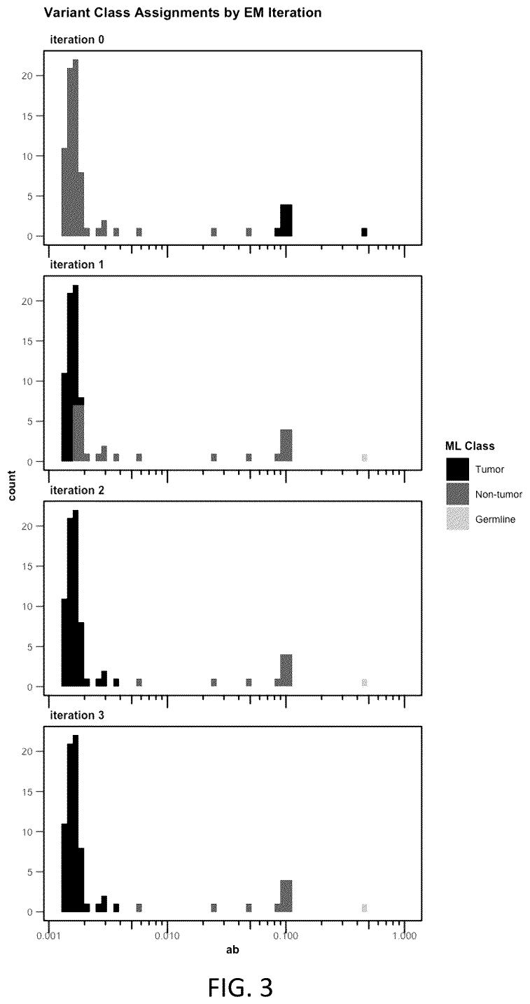

A sample was sequenced and initial analysis of the undiluted cfDNA and buffy normal capture data found that it appeared to have a mix of tumor, CHIP, and germline variants in the target set. The original tumor fraction model, which assumed all variants were from the tumor, fit the cfDNA at 0.05% and the buffy normal at 13.5%. The ctDNA fraction estimated for the sample with a single class model estimated tumor fraction at 0.05%, while the 3-class model estimated tumor fraction at 0.01824%. As shown below, the 3-class model, estimated the tumor fraction at 0.01824% was ran on the cfDNA capture data, first from good (close to truth) starting parameter values (FIG. 2) and then from bad (implausible) starting parameter values (FIG. 3) to show how the EM algorithm iteratively improves model fits. The resulting ctDNA fraction from bad (implausible) starting parameter values as shown in FIG. 3 matches the estimate from good (close to truth) starting parameter values as shown in FIG. 2.

### Example 3: Running EM on the Buffy Normal Negative Control

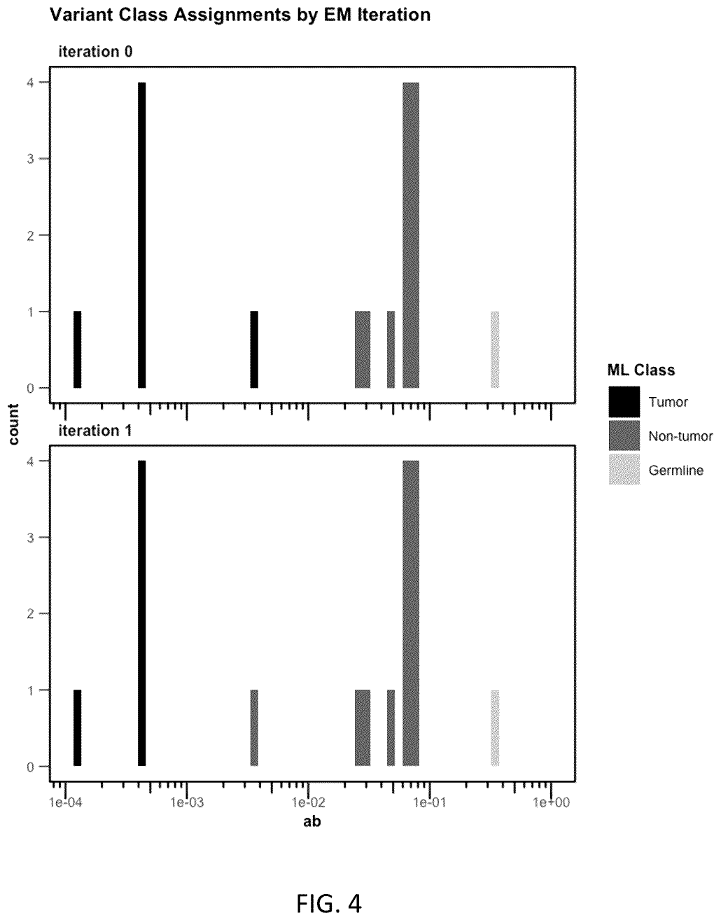

In a model where all sites are assumed to be from the tumor, the buffy normal fit at over 13% tumor fraction, because several CHIP-like sites were detected at nearly 10% allele balance (note this was after applying a filter to drop sites at over 10% allele balance, so this was not caused by germline sites). Below, the 3-class tumor fraction model was run on the buffy normal capture data with no up-front filters on variants passed to the model (FIG. 4). The majority of variant detections were classified as non-tumor as expected given the negative control sample contains no tumor DNA.

### Example 4: The Null Model

The null model is that tumor fraction is zero and all target detections are either errors, CHIP mutations, or germline. The null likelihood can be calculated by setting t_{f}=0, keeping π from the ML fit on the full data, and calculating the total likelihood.

### Example 5: MRD Calling

The ratio of likelihoods for the maximum-likelihood model fit vs the null model fit can be used as a statistic to call positive or negative for MRD.

$$L R = \frac{arg max ( L ( X ) )}{L ( X \vert {t}_{f} = 0 )}$$

Or in log space, it is the difference in log likelihoods:

$$log ( L R ) = log ( arg max ( L ( X ) ) - log ( L ( X \vert {t}_{f} = 0 )$$

Ideally the negative control LLR would be negative (or zero), but here a few variants remain in the “tumor” class at very low allele balance. Still, it is two orders of magnitude less than the cfDNA (and significantly improved compared to the original one-class model fit, which had an LLR of over 2000 on the buffy negative control). By running a large number of negative controls, it is possible to establish a reasonable cutoff at which a positive can be called.

### Example 6: Tumor Fraction Estimate Accounting for Copy Number, Dropout Rate, and Mutation Specific Error Rate

A pair of matched tumor and normal cell lines were analyzed by whole genome sequencing and somatic variant targets were identified. Oligonucleotide probes targeting the identified variants were manufactured. The tumor and normal cell line DNA was sheared to mimic cell-free DNA and mixed at various ratios to obtain tumor DNA concentrations ranging from 0.005% to 1%. These prepared samples, along with the tumor and normal control DNA, underwent sequencing library preparation, unique molecular identifiers (UMIs) were attached, hybridization capture using the probe panel was performed, followed by targeted sequencing. Sequence data was deduplicated using the combined family read approach. Count data of alleles at each target site were gathered and data was analyzed using a likelihood model to estimate the tumor fraction of the sample. The analysis was continuously repeated by applying a resampling approach, selecting a subset of the total targets to generate a smaller panel. This subsampling approach was applied 100 times for each sample with the following panel sizes (number of targets): 16, 50, 100, 150.

The likelihood model represents the number of alternate (alt) allele counts for a target as a binomial distribution, B(n, p), where n is the total number of counts observed at a target site and p is the probability of observing and alt count. Here, p is a function of the tumor DNA fraction (TF), tumor and normal copy number states (CN_{T} and CN_{N}), allele balance of the target in the tumor (AB_{T}), purity of the tumor sample (TP) and error (err).

$$p = \frac{T F \cdot {CN}_{T}}{T F \cdot {CN}_{T} + ( 1 - T F ) \cdot {CN}_{N}} \cdot {AB}_{T} / T P + err$$

Likelihood for each target was aggregated into a mixture model, where DR represents a “dropout rate” that is a function of a false discovery rate of target somatic variants. For example, when a target is incorrectly identified as a somatic variant, such as, a biological dropout, that can be due to biological reasons, for example, the failure of a somatic variant to shed into plasma due to biological features such as sub-clonal structure of the tumor.

$$D R = F D R + ( 1 - F D R ) \cdot P D R$$

The dropout rate (DR) is used to weight a binomial mixture model of the base model+an error-only model:

$$arg max [ {\prod }_{i = 1}^{j} ( 1 - D R ) L ( {p}_{i} \vert {n}_{i} , {k}_{i} ) + D R \cdot L ( err \vert {n}_{i} , {k}_{i} ) ] ,$$

$$where : L ( p \vert n , k ) = P r ( n , k \vert p ) and k = ALT counts$$

Whereby a higher likelihood ratio indicates a greater confidence in tumor recurrence:

$$L R = \frac{L ( p \vert n , k )}{L ( e r r \vert n , k )}$$

CN_{N} was assumed to be 2 and TP was assumed to be 1. CN_{T} and AB_{T} were obtained by analysis of tumor DNA by whole genome sequencing. Analysis was performed using two dropout rates: 1) zero, assuming that no targets are falsely identified and 2) 10%, consistent with 21/240 targets from the example panel having no alt allele counts in the captured tumor DNA data. Additionally, analysis was performed using different error estimates. First, err was set to an arbitrarily low value of 10-9 for calculations. Second, the error rate for each mutation type (e.g., C>T or T>A) was calculated by extracting alt and total counts for control sites (i.e., all sites within 120 bases of the target site that had alternate allele frequencies<1%) and determining a cumulative allele frequency for each possible mutation type. Alternatively, a maximum likelihood estimate using a binomial model for counts for each control site for each mutation type was applied. The error rate matching the mutation type for each target was used in the model above.

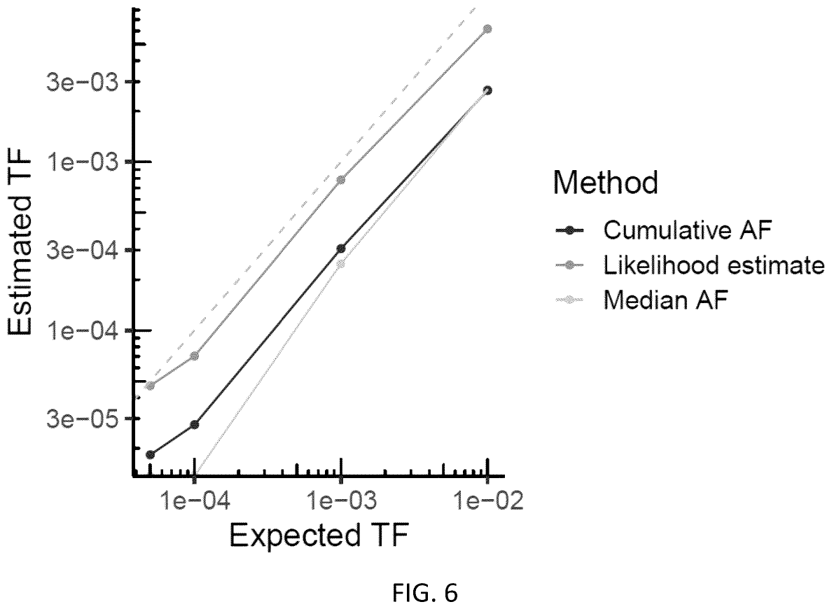

Comparison of tumor fraction estimates: The tumor fraction (TF) estimate resulting from calculation using the model described above (likelihood model) with a dropout rate of 10% and sample-specific, mutation-specific error rates derived from control site data were compared to two alternative methods of determining tumor fraction (FIG. 6). The first alternative method uses the median allele fraction (AF) for all targets in the panel as an estimate for the tumor fraction. This method performed particularly poorly at a low TF as most targets had a 0 alt counts, which resulted in the median estimate for TF to be 0. The second alternative method used the cumulative AF across all targets in the panel, i.e., the sum of all alt counts divided by the total number of counts observed on panel targets. This method is more robust for low TF, but provides a significant underestimation of TF relative to the likelihood-based mixture model. This underestimation is largely caused by not accounting for the zygosity and copy number of the target somatic variant. For example, if tumor DNA is present in a sample of cfDNA for a target that is copy number two, the observed AF will be lower if the target somatic variant was heterozygous in the tumor rather than homozygous alt. In this particular case, the AF-based TF will be half of the true AF if the zygosity is not accounted for.

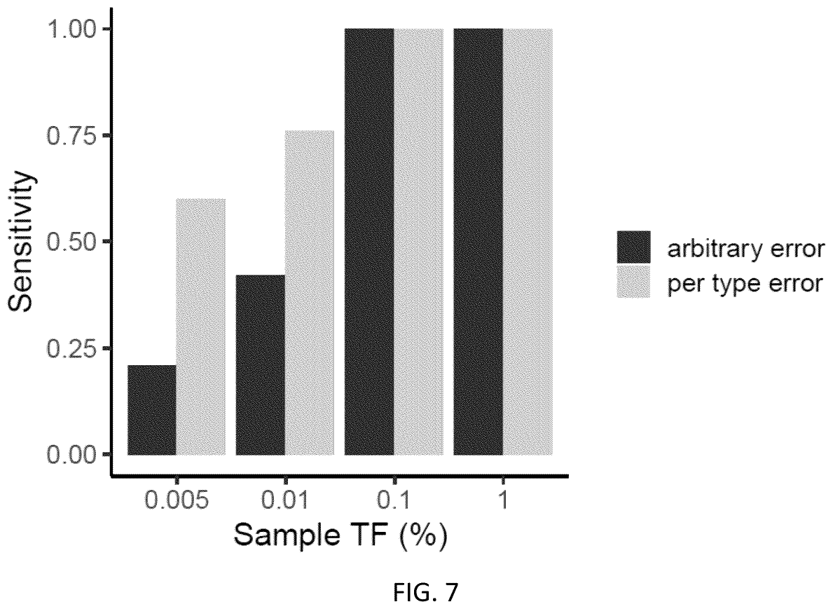

Impact on sensitivity using error rates: Error rates play a key role in determining sensitivity of the assay. Error occurs in all samples, regardless of how much tumor DNA is present, even in a sample with no tumor DNA (i.e., a blank or normal sample). A set of blank samples was used to determine a detection threshold or Limit of Blank. The threshold for positivity was set at the measured value for which 95% of blank samples are negative. The log likelihood ratio was used as a measure of positivity and a threshold based on the 95 percentile value across 100 simulated panels assayed on normal target capture sequencing data was set. FIG. 7 shows results for 16 targets across each TF. The use of error rates derived from control sites (per mutation type error rates) improved the sensitivity of the assay relative to using an arbitrarily low error rate. This effect is more apparent with lower panel sizes and lower TF, where detection is generally more challenging and spurious/erroneous alt allele counts have a more profound impact. In this case, use of sample and target-specific error rates approximately doubles sensitivity in the most challenging case.

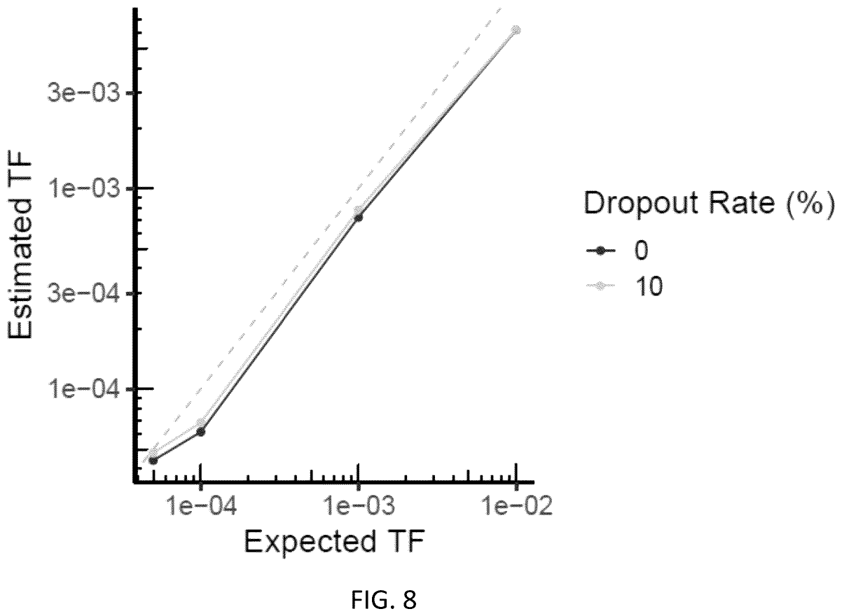

Impact of TF using dropout rates: When targets are falsely identified and included on a panel, those targets can suppress the estimate of TF if all targets are assumed to be real, shed somatic variants. This occurs because a false or unshed somatic variant will not be detected in cfDNA above baseline error levels. With no alt counts, the TF for the sample will tend to fit (have the highest likelihood) at a slightly lower level, roughly proportional to the number of false targets. TF estimated from models fit with 0% DR or 10% DR were compared (FIG. 8). Inclusion of a dropout rate consistent with the false positive somatic target rate results in TF estimates that are closer to the expected value. For the 0.01% TF sample, use of a 10% dropout rate results in TF estimates that are approximately 11% higher than with no dropout modeled.

It is understood that the examples and embodiments described herein are for illustrative purposes only and that various modifications or changes in light thereof will be suggested to persons skilled in the art and are to be included within the spirit and purview of this application and scope of the appended claims. Therefore, the description should not be construed as limiting the scope of the invention.

All publications, patents, and patent applications cited herein are hereby incorporated by reference in their entireties for all purposes and to the same extent as if each individual publication, patent, or patent application were specifically and individually indicated to be so incorporated by reference.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds, compositions or biological systems, which can, of course, vary. It is also to be understood that the terminology used herein is for the purpose of describing particular embodiments only and is not intended to be limiting.

All patents, patent applications, provisional applications, and publications referred to or cited herein are incorporated by reference in their entirety, including all figures and tables, to the extent that are not inconsistent with the explicit teachings of this specification.

What is claimed is:

1. A method of detecting circulating tumor DNA (ctDNA) in a sample, comprising:
(a) obtaining a tumor sample and a non-tumor sample from a cancer patient;
(b) sequencing DNA from the tumor sample and sequencing DNA from the non-tumor sample, thereby obtaining sequences of DNA from the tumor sample and sequences of DNA from the non-tumor sample;
(c) determining somatic variants based on differences between sequences of DNA from the tumor sample and sequences of DNA from the non-tumor sample;
(d) at a later time, obtaining at least one further sample of blood, plasma, or serum from the cancer patient;
(e) extracting cell-free DNA (cfDNA) from the at least one further sample;
(f) sequencing the cfDNA, thereby obtaining a sequencing library; and
(g) detecting the presence or absence of ctDNA sequences in the sequencing library, wherein detecting the presence or absence of ctDNA sequences in the sequencing library comprises:
(i) calculating, using a computer processor, an allele balance of the somatic variants based on purity of the tumor sample obtained from the subject,
(ii) calculating, using a computer processor, a copy number for each somatic variants,
(iii) correcting for, using a computer processor, an error rate for each somatic variant, or
(iv) any combination thereof.

2. The method of claim 1 further comprising calculating, using a computer processor, a dropout rate of sites that do not contribute to a ctDNA signal.

3. The method of claim 1 further comprising correcting for, using a computer processor, inclusion of latent variant classes in the sequencing library by comparing the sequence library to a reference panel (e.g., known buffy coat sequences).

4. A method of detecting circulating tumor DNA (ctDNA) in a sample, comprising:
(a) obtaining a tumor sample and a non-tumor sample from a cancer patient;
(b) sequencing DNA from the tumor sample and sequencing DNA from the non-tumor sample, thereby obtaining sequences of DNA from the tumor sample and sequences of DNA from the non-tumor sample;
(c) determining somatic variants based on differences between sequences of DNA from the tumor sample and sequences of DNA from the non-tumor sample;
(d) at a later time, obtaining at least one further sample of blood, plasma, or serum from the cancer patient;
(e) extracting cell-free DNA (cfDNA) from the at least one further sample;
(f) sequencing the cfDNA, thereby obtaining a sequencing library;
(g) selecting a control site in the sequence library for each somatic variant, wherein the control site matches a reference base for the corresponding somatic variant;
(h) calculating, using a computer processor, an error rate for a variant allele of the reference base by detecting changes at the control site, wherein the error rate is a background probability of detecting the variant allele; and
(i) detecting the presence or absence of ctDNA sequences in the sequencing library.

5. The method of claim 4, wherein the control site is located within about 160 bases of the corresponding somatic variant.

6. The method of claim 4, wherein the control site is located within about 120 bases of the corresponding somatic variant.

7. The method of claim 4, wherein the control site is located within about 20 bases of the somatic variant.

8. The method of claim 4, wherein the control site is located within about 3 bases of the corresponding somatic variant.

9. The method of claim 1, further comprising enriching the extracted cfDNA prior to sequencing by contacting the extracted cfDNA with a plurality of oligonucleotides, wherein each oligonucleotide in the plurality of oligonucleotides comprises a nucleic acid sequence that is capable of hybridizing to a DNA fragment comprising one of the somatic variants, thereby obtaining a ctDNA-enriched fraction;
optionally wherein the enriching comprises (i) hybrid capture-based enrichment, (ii) PCR-target enrichment, or (iii) on-sequencer enrichment.

10. The method of claim 1, wherein the sequencing comprises whole genome sequencing or targeted sequencing.

11. The method of claim 10, wherein the targeted sequencing comprises sequencing of introns, exons, intergenic regions, or a combination thereof.

12. The method of claim 1, wherein the somatic variants comprises at least 10, at least 50, at least 100, at least 150, at least 200, at least 250, or at least 500 tumor-specific somatic mutations.

13. The method of claim 1, wherein the somatic variants comprises one or more somatic mutations selected from SNVs, insertions, deletions, and translocations.

14. The method of claim 1 further comprising determining a tumor fraction.

15. The method of claim 11, wherein a tumor fraction of zero indicates the absence of the tumor in the patient.

16. The method of claim 1, wherein the tumor sample comprises a solid tumor biopsy or a fluid sample.

17. The method of claim 16, wherein the fluid sample is selected from blood, blood plasma, blood serum, urine, saliva, and cerebral spinal fluid (CSF).

18. The method of claim 1, wherein the non-tumor sample comprises a tissue sample matched to a tissue of origin of the tumor sample.

19. The method of claim 1, wherein the non-tumor sample comprises a fluid sample selected from a buffy coat sample, blood, blood plasma, blood serum, urine, saliva, and cerebral spinal fluid (CSF).

20. The method of claim 1, wherein the patient has completed at least one cancer treatment prior to obtaining the tumor sample and the non-tumor sample.

21. The method of claim 20, wherein the cancer treatment is selected from chemotherapy, radiotherapy, surgery, immunotherapy, cell therapy, or biologic therapy.

22. The method of claim 1 further comprising repeating (d)-(g) with a second, third, fourth, fifth, sixth, seventh, eight, nineth, or tenth further sample of blood, plasma, or serum at successive time points.

23. The method of claim 22, wherein (d)-(g) are repeated one or more times while the patient is in remission.

24. The method of claim 22, wherein (d)-(g) are repeated one or more times while the patient is undergoing treatment for the cancer.

25. The method of claim 22, wherein (d)-(g) are repeated one or more times coinciding with or prior to surgery; following, during, or prior to administration of chemotherapy; following, during, or prior to radiation therapy; following, during, or prior to administration of an immunotherapy; following, during, or prior to administration of a cell therapy; or following, during, or prior to administration of a biologic therapy.

26. The method of claim 1, wherein the tumor is selected from adrenal cancer, anal cancer, bile duct cancer, bladder cancer, bone cancer, a brain/CNS tumor, breast cancer, Castleman disease, cervical cancer, colon or rectum cancer, endometrial cancer, esophagus cancer, a Ewing tumor, eye cancer, gallbladder cancer, a gastrointestinal carcinoid tumor, a gastrointestinal stromal tumor (GIST), gestational trophoblastic disease, Hodgkin disease, Kaposi sarcoma, kidney cancer, laryngeal and hypopharyngeal cancer, leukemia, liver cancer, lung cancer, lymphoma, malignant mesothelioma, multiple myeloma, myelodysplastic Syndrome, nasal cavity or paranasal sinus cancer, nasopharyngeal cancer, neuroblastoma, oral cavity or oropharyngeal cancer, osteosarcoma, ovarian cancer, pancreatic cancer, penile cancer, a pituitary tumor, prostate cancer, retinoblastoma, rhabdomyosarcoma, salivary gland cancer, skin cancer, small intestine cancer, stomach cancer, testicular cancer, thymus cancer, thyroid cancer, uterine sarcoma, vaginal cancer, vulvar cancer, Waldenstrom macroglobulinemia, and Wilms tumor.
